(19) United States
(12) Patent Application Publication
(10) Pub. No.: US 2026/0242352 A1
(43) Pub. Date: Aug. 20, 2026

(54) INHIBITION OF SHIP1 AS A THERAPEUTIC STRATEGY FOR THE TREATMENT OF ALZHEIMER'S DISEASE

(71) Applicants: The Trustees of Indiana University, Bloomington, IN (US); Purdue Research Foundation, West Lafayette, IN (US)

(72) Inventors: Timothy Ivo Richardson, Zionsville, IN (US); Cynthia Darshini Jesudason, Indianapolis, IN (US); William Brent Clayton, Brownsburg, IN (US)

(21) Appl. No.: 18/993,975

(22) PCT Filed: Jul. 11, 2023
(86) PCT No.: PCT/US23/69922

§ 371 Date: Jan. 13, 2025

Related U.S. Application Data
(60) Provisional application No. 63/389,039, filed on Jul. 14, 2022.

(51) Int. Cl. C07D 401/14; A61K 31/454; A61K 31/4545; A61P 25/28; C07D 401/04

(52) U.S. Cl. CPC C07D 401/14; A61K 31/454; A61K 31/4545; A61P 25/28; C07D 401/04

(57) ABSTRACT

The general field of the present disclosure are novel approaches to the treatment of Alzheimer's and other neurodegenerative disorders using novel therapeutics comprising SHIP1 phosphatase inhibitors. Specifically, the disclosure provides compounds, pharmaceutical compositions and methods of treating and preventing such disorders.

### CROSS-REFERENCE TO RELATED APPLICATION

This application claims the benefit of U.S. Provisional Patent App. No. 63/389,039 filed Jul. 14, 2022, the content of which is hereby incorporated by reference herein in its entirety.

### REFERENCE TO GOVERNMENT GRANTS

This invention was made with government support under National Institutes of Health awarded by AG065181. The Government has certain rights in the invention.

### FIELD

The general field of the present disclosure are novel approaches to the treatment of Alzheimer's and other neurodegenerative disorders using novel therapeutics comprising SHIP1 phosphatase inhibitors

### BACKGROUND

Alzheimer's disease (AD) is a fatal, neurodegenerative disorder, characterized by histopathological accumulation of extracellular β-amyloid (Aβ) plaques and intra-neuronal neurofibrillary tangles (NFTs), which have been hypothesized to result in neurotoxicity and progressive cognitive decline. See Kumar et al., “A review on Alzheimer's disease pathophysiology and its management: an update, (2015) Pharmacol Rep 67: pp. 195-203; Hardy et al., “Alzheimer's disease: the amyloid cascade hypothesis: an update and reappraisal,” (2006) J Alzheimers Dis 9: pp. 151-153; Shen et al, “Complement activation by neurofibrillary tangles in Alzheimer's disease,” (2001) Neurosci Lett 305: pp. 165-168; Scheltens et al., “Alzheimer's disease, (2021) Lancet 397: pp. 1577-1590. The amyloid cascade hypothesis postulates that various forms of Aβ oligomers and plaques are instrumental in a neuropathological process that triggers subsequent NFT pathology, neuroinflammation, and neuronal loss; however, the mechanisms by which Aβ influences neurotoxic signaling including NFT formation remain an area of intense study. Recently, the amyloid cascade hypothesis has come under increased scrutiny due to failures of drugs targeting Aβ peptide processing and Aβ plaque. See Panza et al., “A critical appraisal of amyloid-beta-targeting therapies for Alzheimer disease,” (2019) Nat Rev Neurol 15: pp. 73-88.

More recent evidence including genome-wide association studies (GWAS) and differential gene expression comparing normal to affected Alzheimer's brain tissue have identified risk and protective variants in genes such as TREM2, CD33, APOE, Aβ CA7, PLCG2, and INPP5D, which are essential to microglia function. See Malik et al., “Genetics ignite focus on microglial inflammation in Alzheimer's disease,” (2015) Mol Neurodegener 10: p. 52. Microglia are the non-neuronal, macrophage-like cells that serve as resident immune cells in the brain. See Vaughan et al., “Neuroglial cells in the cerebral cortex of rats from young adulthood to old age: an electron microscope study,” (1974) J Neurocytol 3: pp. 405-429.

During development microglia originate from stem cells in the yolk sac and differentiate into CD45⁺, CX3CR1⁺ immune cells that migrate to the CNS. See Kierdorf et al., “Microglia emerge from erythromyeloid precursors via Pu.1- and Irf8-dependent pathways,” (2013) Nat Neurosci 16: pp. 273-280. Once resident, these cells renew slowly in humans at a rate of approximately 28% per year, thus providing a mechanism to renew microglia. See Reu et al., “The Lifespan and Turnover of Microglia in the Human Brain,” (2017) Cell Rep 20: pp. 779-784. Disease associated microglia (DAM) have been characterized at sites of Aβ plaques and neurodegeneration in animal models. See Keren-Shaul et al., “A Unique Microglia Type Associated with Restricting Development of Alzheimer's Disease,” (2017) Cell 169: pp. 1276-1290. Although their relevance to human microglia in AD remains a current area of intense study, they have gene signatures associated with lipid metabolism and phagocytosis hypothesized to reflect the neuroprotective role of microglia in the clearance of extracellular toxins. See Olah et al., “Single cell RNA sequencing of human microglia uncovers a subset associated with Alzheimer's disease,” (2020) Nat Commun 11: pp. 6129. A two-state model of DAM induction has been proposed, in which homeostatic microglia that are associated with and support the health of neurons become activated with increased expression of DAP12, APOE, and Triggering receptor expressed on myeloid cells-2 (TREM2). TREM2 ligands such as apolipoproteins (APOEs) and Aβ induce microglial differentiation into stage 2 DAMs with increased expression of LP1, CST7, and AXL. Deczkowska et al., “Disease-Associated Microglia: A Universal Immune Sensor of Neurodegeneration,” (2018) Cell 173: pp. 1073-1081; Keren-Shaul et al. 2017.

TREM2 is a receptor expressed on the surface of microglia. Genetic evidence suggests that lower TREM2 expression and inactivating variants increase risk of AD. See Jonsson et al., “Variant of TREM2 associated with the risk of Alzheimer's disease,” (2013) N Engl J Med 368: pp. 107-116. TREM2 binds Aβ and APOE, which activates microgliosis and the clearance of extracellular debris. See Yeh et al., “TREM2 Binds to Apolipoproteins, Including APOE and CLU/APOJ, and Thereby Facilitates Uptake of Amyloid-Beta by Microglia,” (2016) Neuron 91: pp. 328-340. The TREM2^{R47H} variant reduces the affinity of TREM2 ligands and cellular activation downstream, which requires DAP12, an adapter protein on the intracellular side of the plasma membrane that associates with numerous signal transduction mediators. See Sudom et al., “Molecular basis for the loss-of-function effects of the Alzheimer's disease-associated R47H variant of the immune receptor TREM2,” (2018) J Biol Chem 293: pp. 12634-12646. For example, recruitment of SYK to phosphorylated tyrosine residues at the C-terminus of DAP12 mediates signaling through phosphorylation of PLCγ2 and subsequent formation of intracellular IP₃ and Ca²⁺ release. See Konishi et al., “Microglial TREM2/DAP12 Signaling: A Double-Edged Sword in Neural Diseases,” (2018) Front Cell Neurosci 12: p. 206. Importantly, an activating variant of PLCG2, PLCG2^{P522R} is protective in AD. See Magno et al., “Alzheimer's disease phospholipase C-gamma-2 (PLCG2) protective variant is a functional hypermorph,” (2019) Alzheimer's Res Ther 11: p. 16. This human genetic evidence suggests that dampened microglia activity increases risk of neurodegeneration while activated microglia are protective, clearing Aβ oligomers and plaques, and mitigating an inflammatory microenvironment that is toxic to neurons. See Deczkowska et al., 2018.

The recent approval of the anti-amyloid antibody Aducanumab provides evidence for the role of activated microglia in the treatment of AD. See Dunn et al., “Approval of Aducanumab for Alzheimer Disease-The FDA's Perspective,” (2021) JAMA Intern Med 181: pp. 1276-1278. Cell surface Fcγ receptors (FcγRs) on microglia recognize the Fc portion of IgG antibodies triggering downstream effector functions. See Bournazos et al., “The role of IgG Fc receptors in antibody-dependent enhancement,” (2020) Nat Rev Immunol 20: pp. 633-643. The clearance of Aβ deposits by Aducanumab has been demonstrated to occur by targeting Aβ via the variable region of the antibody coupled to FcγR-mediated enhancement of microglia recruitment and phagocytosis. See Sevigny et al., “The antibody aducanumab reduces Abeta plaques in Alzheimer's disease,” (2016) Nature 537: pp. 50-56.

The INPP5D gene encodes the Src homology 2 (SH2) domain-containing phosphatase-1 (SHIP1), which is a phosphatidylinositol phosphatase that plays a key role regulating pathways downstream from TREM2. See Peng et al., “TREM2- and DAP12-dependent activation of PI3K requires DAP10 and is inhibited by SHIP1,” (2010) Sci Signal 3: p. 38; Pauls et al., “Regulation of immune cell signaling by SHIP1: A phosphatase, scaffold protein, and potential therapeutic target,” (2017) Eur J Immunol 47: pp. 932-945. SHIP1 is a complex, multi-domain protein with a phosphatase (Ptase) domain flanked by a pleckstrin-homology (PH) domain that binds phosphatidylinositol (3,4,5)-trisphosphate [PI(3,4,5)P₃] and a C2 domain that binds phosphatidylinositol (3,4)-bisphosphate [PI(3,4)P₂]. See Damen et al., “The 145-kDa protein induced to associate with Shc by multiple cytokines is an inositol tetraphosphate and phosphatidylinositol 3,4,5-triphosphate 5-phosphatase,” (1996) Proc Natl Acad Sci USA 93: pp. 1689-1693; Blunt et al., “Pharmacological targeting of phosphoinositide lipid kinases and phosphatases in the immune system: success, disappointment, and new opportunities,” (2012) Front Immunol 3: p. 226. The PH and C2 domains locate and orient the catalytic site towards its PI(3,4,5)P₃ substrate at the intracellular side of the membrane. The C2 domain is essential for cellular function and interactions between the Ptase and C2 domains modulate enzymatic activity. See Le Coq et al., “Structural basis for interdomain communication in SHIP2 providing high phosphatase activity,” (2017) Elife 6: p. 26640. SHIP1 converts PI(3,4,5)P₃ to PI(3,4)P₂. SHIP1 also contains an N-terminal SH2 domain that binds immunoreceptor tyrosine-based activation motifs (ITAMs) and a C-terminal proline rich domain that binds many other proteins including PLCγ2 and the Tec and Syk family kinases. PI(3,4,5)P₃ binds and activates other PH-containing proteins such as PLCγ2, PDK1, and AKT. See Scheffzek et al., “Pleckstrin homology (PH) like domains-versatile modules in protein-protein interaction platforms,” (2012) FEBS Lett 586: pp. 2662-2673. Because SHIP1 binds ITAMs, competes with kinases, and converts PI(3,4,5)P₃ to PI(3,4)P₂, it limits downstream signaling in multiple ways, and is therefore understood as a brake on microglia activation. See Pauls et al. 2017.

Taken together this understanding of AD risk and protective variants in genes critical to the activity of microglia, the role of microglia in the clearance of Aβ, and SHIP1 as a limiting node downstream from TREM2 and FCγRIIB, suggests that inhibition of SHIP1 would activate microglia, and would therefore be an effective therapeutic strategy in disease. We also anticipate that this therapeutic intervention could be synergistically combined with anti-amyloid and/or TREM2 agonist antibodies. Therefore, inhibitors of SHIP1 would increase the protective functions of microglia and could therefore be used to prevent or treat disease, reduce the rate of disease progression and cognitive decline in patients, and reverse neurodegeneration.

The present disclosure provides novel compounds that are SHIP1 inhibitors that address the need for a potent and effective treatment for Alzheimer's Disease and Alzheimer's Disease-related Dementias. The present disclosure also provides a pharmaceutical composition for the prevention of Alzheimer's Disease and Alzheimer's Disease-related Dementias.

### SUMMARY

Recent evidence including GWAS and differential gene expression comparing normal to affected Alzheimer's brain tissue have identified risk and protective variants in genes such as TREM2, PLCG2 and INPP5D that are essential to microglia function. INPP5D encodes SHIP1, a multi-domain protein with a phosphatase that converts PI(3,4,5)P3 to PI(3,4)P2, a SH2 domain that interacts with receptor ITAMs and competes with SYK, and a proline rich region that binds many other proteins. SHIP1 therefore limits microglia activation in multiple ways. Inhibition of SHIP1 early in disease would increase microglial protective functions and reduce the rate of disease progression and cognitive decline in Alzheimer's patients.

The inventors have performed a screen of 50K compounds at the SHIP1 phosphatase, analyzed a publicly available fragment-based screen, and evaluated inhibitors reported in the literature. They utilized the malachite green assay with PtdIns(3,4,5)P3-diC8 and SHIP1 Ptase-C2 to measure inhibitory potency. A Cellular Thermal Shift Assay was used to confirm target engagement in cells. A phosphoAKT assay was used to provide additional evidence of on-target activity. A high-content imaging assay measuring phagocytosis, cell number, and nuclear intensity was implemented using the BV2 and HMC3 cell lines to characterize cellular pharmacology and cytotoxicity. Mouse microglia were assayed to demonstrate similar activity in primary cells. Inhibitors predicted to have drug-like properties were subjected to assays measuring solubility, cellular permeability, and mouse microsomal stability. A physiological based pharmacokinetic model was compared to measured exposure in vivo for select compounds upon oral administration in mice.

Inhibition of SHIP1 is a novel therapeutic strategy for treatment of Alzheimer's. Identified were structurally distinct molecular scaffolds with varying degrees of enzyme inhibition, cellular activity, and exposure in mice.

More specifically, the present disclosure provides a compound of formula I.

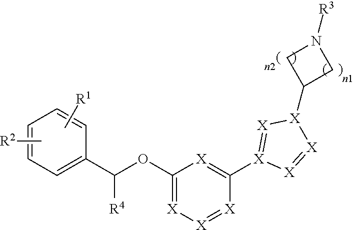

each X is independently C or N;

$$n 1 + n 2 = 4 ;$$

R1, R2 are independently H, F, Cl, Br or methyl;
R3 is H, amide, carbamate, or alkyl; and
R4 is H or CH₃,
or pharmaceutically acceptable salt thereof.

In other embodiments, the present disclosure encompasses a method for treating Alzheimer's disease in a patient, which comprises administering to a patient in need thereof a compound of the present disclosure or a pharmaceutically acceptable salt thereof. Furthermore, the present disclosure encompasses a method for treating Alzheimer's disease-related dementias in a patient, comprising administering to a patient in need thereof a compound of the present disclosure or a pharmaceutically acceptable salt thereof. The present disclosure further provides a method of treating the progression of mild cognitive impairment to Alzheimer's disease or related dementias in a patient, comprising administering to a patient in need of such treatment an effective amount of a compound of the present disclosure. The present disclosure further provides a method of preventing Alzheimer's disease or Alzheimer's disease-related dementias, comprising administering to a patient in need of such treatment an effective amount of a compound of the present disclosure.

The present disclosure provides a pharmaceutical composition comprising a compound of the disclosure, or a pharmaceutically acceptable salt thereof, in combination with one or more pharmaceutically acceptable carriers, diluents or excipients. In another embodiment, the composition further comprises one or more additional therapeutic agents. In a further embodiment, the present disclosure provides a pharmaceutical composition for the treatment of Alzheimer's disease, comprising a compound of the disclosure, or a pharmaceutically acceptable salt thereof, in combination with one or more pharmaceutically acceptable carriers, diluents or excipients. In yet another embodiment, the present disclosure provides a pharmaceutical composition for the treatment of Alzheimer's disease-related dementias, comprising a compound of the disclosure, or a pharmaceutically acceptable salt thereof, in combination with one or more carriers, diluents, or pharmaceutically acceptable excipients. In yet another embodiment, the present disclosure provides a pharmaceutical composition for the prevention of Alzheimer's disease and Alzheimer's disease-related dementias, comprising a compound of the disclosure, or a pharmaceutically acceptable salt thereof, in combination with one or more carriers, diluents, or pharmaceutically acceptable excipients.

Furthermore, the present disclosure provides a compound of the disclosure, or a pharmaceutically acceptable salt thereof, for use in therapy, in particular for the treatment of Alzheimer's disease. Furthermore, the present disclosure provides a compound of the disclosure, or a pharmaceutically acceptable salt thereof, for use in the treatment of Alzheimer's disease. In a further embodiment, the present disclosure provides the use of a compound of the disclosure, of or a pharmaceutically acceptable salt thereof, for the manufacture of a medicament for the treatment of Alzheimer's disease.

In other embodiments, the present disclosure provides a compound of the disclosure, or a pharmaceutically acceptable salt thereof, for use in therapy, in particular for the treatment of Alzheimer's disease-related dementias. Furthermore, the present disclosure provides a compound of the disclosure, or a pharmaceutically acceptable salt thereof, for use in the treatment of Alzheimer's disease-related dementias. In a further embodiment, the present disclosure provides the use of a compound of the disclosure, of or a pharmaceutically acceptable salt thereof, for the manufacture of a medicament for the treatment of Alzheimer's disease-related dementias.

Furthermore, the present disclosure provides a compound of the disclosure, or a pharmaceutically acceptable salt thereof, for use in the prevention of Alzheimer's disease and Alzheimer's disease-related dementias. Furthermore, the present disclosure provides a compound of the disclosure, or a pharmaceutically acceptable salt thereof, for use in the prevention of Alzheimer's disease and Alzheimer's disease-related dementias. In a further embodiment, the present disclosure provides the use of a compound of the disclosure, of or a pharmaceutically acceptable salt thereof, for the manufacture of a medicament for use in the prevention of Alzheimer's disease and Alzheimer's disease-related dementias.

The present disclosure also encompasses intermediates and processes useful for the synthesis of a compound of the present disclosure.

Alzheimer's disease-related dementias (ADRD) include Lewy body dementia (LBD), frontotemporal degeneration (FTD), vascular cognitive impairment and dementia (VCID), and multiple etiology dementias. Mild cognitive impairment is defined as the potential prodromal phase of dementia associated with Alzheimer's disease based on clinical presentation and on progression of patients exhibiting mild cognitive impairment to Alzheimer's disease overtime. See Morris et al., “Mild cognitive impairment represents early-stage Alzheimer disease,” (2001) Arch Neurol 58: pp. 397-405; Petersen et al., “Mild cognitive impairment: clinical characterization and outcome,” (1999) Arch Neurol 56: pp. 303-308.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

In some embodiments, the disclosure provides for a composition comprising a SHIP1 inhibitor for use in combination with an antibody or antigen-binding fragment for treating Alzheimer's disease and Alzheimer's disease-related dementias.

### BRIEF DESCRIPTION OF THE DRAWINGS

FIG. 1 is a schematic depicting the SHIP1 protein domains and other signaling proteins downstream from the TREM2 receptor.

FIG. 2 is a schematic depicting the malachite green enzyme assay.

FIG. 3 shows the activity of Compounds 9 and 10 in primary mouse microglia.

FIG. 4 is a graph depicting the pharmacokinetics of Compounds 9 and 10 in C57BL/6J mice.

### DETAILED DESCRIPTION

Various quantities, such as amounts, sizes, dimensions, proportions, and the like, are presented in a range format throughout this disclosure. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is to describe particular embodiments only and is not intended to be limiting of any embodiment. As used herein, the singular forms “a,” “an”, and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless expressly stated or obvious from context, as used herein, the term “about” in reference to a number or range of numbers is understood to mean the stated number and numbers +/−10% thereof, or 10% below the lower listed limit and 10% above the higher listed limit for the values listed for a range.

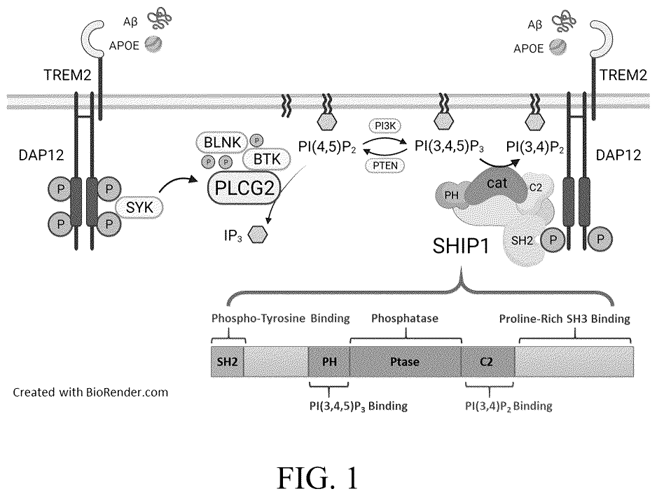

Recent evidence including GWAS and differential gene expression comparing normal to affected Alzheimer's brain tissue have identified risk and protective variants in genes such as TREM2, PLCG2, and INPP5D that are essential to microglia function. The INPP5D gene encodes the Src homology 2 (SH2) domain-containing phosphatase-1 (SHIP1), which is a phosphatidylinositol phosphatase that plays a key role regulating pathways downstream from TREM2. See Peng et al. 2010; Pauls et al., 2017. SHIP1 is a complex, multi-domain protein with a phosphatase (Ptase) domain flanked by a pleckstrin-homology (PH) domain that binds phosphatidylinositol (3,4,5)-trisphosphate [PI(3,4,5)P₃] and a C2 domain that binds phosphatidylinositol (3,4)-bisphosphate [PI(3,4)P₂]. See FIG. 1; Damen et al. 1996; Blunt et al., 2012. The PH and C2 domains locate and orient the catalytic site towards its PI(3,4,5)P₃ substrate at the intracellular side of the membrane. The C2 domain is essential for cellular function and interactions between the Ptase and C2 domains modulate enzymatic activity. See Le Coq et al., 2017. SHIP1 converts PI(3,4,5)P₃ to PI(3,4)P₂. SHIP1 also contains an N-terminal SH2 domain that binds immunoreceptor tyrosine-based activation motifs (ITAMs) and a C-terminal proline rich domain that binds many other proteins including PLCγ2 and the Tec and Syk family kinases. PI(3,4,5)P₃ binds and activates other PH-containing proteins such as PLCγ2, PDKI, and AKT. See Sscheffzek et al. 2012. Because SHIP1 binds receptor ITAMs, competes with kinases, and converts PI(3,4,5)P₃ to PI(3,4)P₂, it limits downstream signaling in multiple ways, and is therefore understood as a brake on microglia activation. Therefore, our therapeutic hypothesis is that inhibition of SHIP1 early in disease would increase microglial protective functions and reduce the rate of disease progression and cognitive decline in Alzheimer's patients.

In any of the embodiments disclosed herein, the term “treating the progression of mild cognitive impairment to Alzheimer's disease” includes restraining, slowing, stopping, or reversing the progression of mild cognitive impairment to Alzheimer's disease in a patient.

In any of the embodiments disclosed herein, the terms “treating” or “to treat” includes restraining, slowing, stopping, or reversing the progression or severity of an existing symptom or disorder.

In any of the embodiments disclosed herein, the term “patient” refers to a human.

A compound of the present disclosure can react to form pharmaceutically acceptable salts. Pharmaceutically acceptable salts and common methodology for preparing them are well known in the art. See, for example, P. Stahl, et al. Handbook of Pharmaceutical Salts: Properties, Selection and Use (Manual of Pharmaceutical Salts: Properties, Selection and Use), 2nd revised edition (Wiley-VCH, 2011); S M Berge, et al., “Pharmaceutical Salts”, Journal of Pharmaceutical Sciences, Vol. 66, No. 1, January 1977.

### Excipients

Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

### “Effective or Therapeutic Amount”

Effective or therapeutic amounts of the compositions of this disclosure include any amount sufficient to inhibit (e.g., slow or stop) the progression of a neurodegenerative disorder. In some embodiments, effective amounts of the compositions include any amount sufficient to inhibit (e.g., slow or stop) the deterioration of the cognitive function of a patient.

The amount of the active ingredient that may be combined with the optional carrier materials to produce a single dosage form may vary depending upon the host treated and the particular mode of administration. The specific dose level for any particular patient may depend upon a variety of factors, including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

### General Methods and Assays

Malachite Green Enzyme Assay. Enzymatic inhibitory potencies (IC₅₀) were determined using PI(3,4,5)P₃-diC8 as a substrate at 25° C. in 50 mM HEPES buffer (pH7.4, 150 mM NaCl, 2 mM MgCl2). Compounds diluted in DMSO were added to 384-well plates. Enzyme solution was added. After a 20 min incubation period, the reaction was initiated by addition of PI(3,4,5)P₃-diC8. Final compound concentrations ranged from 50 nM to 950 μM. Final reaction concentrations for the PI(3,4,5)P₃-diC8 substrate and the enzyme were 52 μM and 10 nM respectively. The reaction was quenched after 10 min by adding Malachite BioMol Green (Enzo Lifesciences, PA, USA). Plates were then incubated for 30 min at room temperature. Absorbance (620 nm) was measured using a SpectraMax Me5 Microplate Reader (Molecular Devices, LLC, USA). IC₅₀ values were calculated by fitting absorbance versus inhibitor concentration.

Cellular thermal shift assay (CETSA). A split Nano Luciferase assay (SplitLuc CETSA) was used to demonstrate target engagement of SHIP1 inhibitors in a physiologically relevant cellular context by quantifying changes in the thermal stability of a HiBit-labeled full length SHIP1 protein in intact cells. This assay was run in the following two formats with HMC3/HiBit-INPP5D stably transfected cells.

1. Thermal shift: cells were treated with 40 μM compound for 60 min, then heated with a temperature gradient covering 38-52° C. for 3 min before luminescence detection. Mean and standard deviation of control Tm were determined to generate a “Mean+3SD” ΔTm, which was used as threshold to determine a significant ΔTm of compound from control. When the difference of ΔTm of compound treated cells from control Tm>3SD, the compound was considered positive for target engagement, otherwise negative. SD=average standard deviation.

2. Compound dose response: run at target Tm (44.2° C. for SHIP1) with compound dosing from 80 uM or 100 uM with 1:3 serial dilutions to generate an 8 point curve. Cells were treated for 60 min before being heated at target Tm for 3 min before luminescence detection. An AC50 was calculated using a four-parameter logistic curve regression model with change at highest concentration noted when difference from control >3SD, otherwise AC50 marked as “nc” for not calculated.

PhosphoAKT assay. THP1 cells were treated with inhibitors for 90 minutes and then the levels of phosphorylated and total AKT (pAKT/tAKT) were detected using the Perkin Elmer Alpha SureFire Ultra Multiplex PhosphoAKT (S473) kit according to the manufacturer's directions. IC₅₀ values were calculated by fitting the ratio of pATK/tAKT versus inhibitor concentration.

pHrodo-myelin phagocytosis/cell health assay with microglial cells. This 384-well plate high content imaging assay was developed to quantify phagocytosis and cell health simultaneously using either BV2 or HMC3 immortalized microglial cell lines or primary microglia isolated from mouse brain. Cells were cultured in DMEM GlutaMax media (ThermoFisher) containing 10% FBS and Pen-Strep in 37° C. 5% CO₂ incubator.

### Assay Timing

Day 1: Cells were plated (Corning Falcon 384 well Optilux Black and clear bottom plates for imaging) with BV2 at 400 cells/45 μl/well, HMC3 at 600 cells/45 μl/well, or primary at 2000 cell/45 μl/well.

Day 2: Cells were treated with 10× serially diluted compounds in a dose range of 60 μM to 3 nM for 48 hrs at 37° C.

Day 3: Cells were seeded with pHrodo-myelin (for total 20 hrs) 24 hrs after starting compound treatment. The pHrodo-myelin stocks were at 1 mg/ml (protein equivalent) stored in −20° C. or −80° C. freezer. Stocks were thawed and diluted with culture media into 10× seeding solution (50 μg/ml) and added 5 μl/well to 384 well cell plate.

Day 4: Cell staining and imaging. Nuclear staining solution was prepared by adding 1 μl of 10 mg/mL Hoechst-33342 to every 1 ml culture media that will be added to cell plate at 20 μl/well. The final concentration of Hoechst-33342 to cells was about 2.5 μg/ml. Cell plates were incubated for >30 min at 37° C. before imaging. Cell plates were scanned with an ArrayScan automatic high content imaging system using a 10× objective lens, 4 fields/well collected. Three measurements were obtained 1) mean total phagocytosis spot intensity per cell, 2) total cell counts per well, and 3) mean average nuclear intensity per cell for cell health. Apoptotic cells showed nuclear intensity increase (early apoptosis) or decrease (later apoptosis).

Activity of Compound 9 in primary mouse microglia. Cortical tissue from C57BL/6J neonatal mice (P0-P3) was homogenized in Dulbecco's Modified Eagle Medium (DMEM), filtered through 250 and 100 μm mesh, and cultured in Advanced DMEM/F12 supplemented with 10% fetal bovine serum, 1× GlutaMAX and 1× Penicillin/Streptomycin. At 21 days in vitro (DIV), the cultures were subjected to mild trypsinization using 0.083% Trypsin-EDTA in DMEM for 30 mins to detach an intact layer of astrocytes. The microglia attached to the bottom were used as described in pHrodo-myelin phagocytosis assay to measure phagocytosis and cell health. The activity of Compound 10 in primary mouse microglia was obtained in a similar manner to Compound 9.

Pharmacokinetics of Compound 9. Compound 9 was formulated at 5 mg/ml in HPMC (1%)/Tween 80 (0.25%)/purified water. Male C57BL/6J mice aged 8-12 weeks from The Jackson Laboratory were dosed (100 mg/kg, 20 ml/kg) via oral gavage. Plasma exposures at 0.5, 1, 2, 4, 6, 8, 12 hr and terminal at 24 hr were obtained. Brain exposures were obtained at terminal 4 and 24 hrs. The pharmacokinetics of Compound 10 was obtained in a similar manner to Compound 9.

Further reference is made to the following experimental examples.

### EXAMPLES

The following examples are provided for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Compounds

TABLE 1

| Com- | | MS | SHIP1 IC₅₀ | |
|---|---|---|---|---|
| pound | Name | (ES+) | μM (n) | |
| 1 | tert-butyl 4-(4-{5-[(2- | 469.22 | 92.1 | (3) |
| | chlorophenyl)methoxy]pyridin-3-yl}- | | | |
| | 1H-pyrazol-1-yl)piperidine-1- | | | |
| | carboxylate | | | |
| 2 | tert-butyl 4-(4-(5-((2- | 453.38 | 81.9 | (8) |
| | fluorobenzyl)oxy)pyridin-3-yl)-1H- | | | |
| | pyrazol-1-yl)piperidine-1- | | | |
| | carboxylate | | | |
| 3 | tert-butyl 4-(4-(5-((2- | 449.38 | 116 | (4) |
| | methylbenzyl)oxy)pyridin-3-yl)-1H- | | | |
| | pyrazol-1-yl)piperidine-1- | | | |
| | carboxylate | | | |
| 4 | tert-butyl 4-(4-(5- | 435.40 | 90.3 | (4) |
| | (benzyloxy)pyridin-3-yl)-1H- | | | |
| | pyrazol-1-yl)piperidine-1- | | | |
| | carboxylate | | | |
| 5 | tert-butyl 4-(4-(5-((2,6- | 503.30 | 35.7 | (3) |
| | dichlorobenzyl)oxy)pyridin-3-yl)- | | | |
| | 1H-pyrazol-1-yl)piperidine-1- | | | |
| | carboxylate | | | |
| 6 | tert-butyl 4-(4-(5-((4- | 469.22 | 137 | (3) |
| | chlorobenzyl)oxy)pyridin-3-yl)-1H- | | | |
| | pyrazol-1-yl)piperidine-1- | | | |
| | carboxylate | | | |
| 7 | tert-butyl 4-(4-(5-((4- | 515.22 | 57.0 | (4) |
| | bromobenzyl)oxy)pyridin-3-yl)-1H- | | | |
| | pyrazol-1-yl)piperidine-1- | | | |
| | carboxylate | | | |
| 8 | tert-butyl 4-(4-(5-((2,4- | 503.30 | 18.1 | (1) |
| | dichlorobenzyl)oxy)pyridin-3-yl)- | | | |
| | 1H-pyrazol-1-yl)piperidine-1- | | | |
| | carboxylate | | | |
| 9 | 3-((2-chlorobenzyl)oxy)-5-(1- | 369.25 | 302 | (8) |
| | (piperidin-4-yl)-1H-pyrazol-4- | | | |
| | yl)pyridine hydrochloride | | | |
| 10 | 3-((2,4-dichlorobenzyl)oxy)-5-(1- | 403.10 | 286 | (9) |
| | (piperidin-4-yl)-1H-pyrazol-4- | | | |
| | yl)pyridine hydrochloride | | | |
| 11 | 3-((4-chlorobenzyl)oxy)-5-(1- | 369.16 | 109 | (3) |
| | (piperidin-4-yl)-1H-pyrazol-4- | | | |
| | yl)pyridine hydrochloride | | | |
| 12 | 3-((4-bromobenzyl)oxy)-5-(1- | 413.08 | 29.7 | (3) |
| | (piperidin-4-yl)-1H-pyrazol-4- | | | |
| | yl)pyridine hydrochloride | | | |
| 13 | 3-((2,6-dichlorobenzyl)oxy)-5-(1- | 403.20 | 147 | (4) |
| | (piperidin-4-yl)-1H-pyrazol-4- | | | |
| | yl)pyridine trifluoroacetate | | | |
| 14 | 3-(benzyloxy)-5-(1-(piperidin-4-yl)- | 335.35 | 208 | (4) |
| | 1H-pyrazol-4-yl)pyridine | | | |
| | trifluoroacetate | | | |
| 15 | 3-((2-chlorobenzyl)oxy)-5-(1-(1- | 453.35 | 43.3 | (4) |
| | (3,3-dimethylbutyl)piperidin-4-yl)- | | | |
| | 1H-pyrazol-4-yl)pyridine | | | |
| 16 | 1-(4-(4-(5-((2- | 467.37 | 102 | (4) |
| | chlorobenzyl)oxy)pyridin-3-yl)-1H- | | | |
| | pyrazol-1-yl)piperidin-1-yl)-3,3- | | | |
| | dimethylbutan-1-one | | | |
| 17 | Methyl 4-(4-(5-((2- | 427.30 | 133 | (3) |
| | chlorobenzyl)oxy)pyridin-3-yl)-1H- | | | |
| | pyrazol-1-yl)piperidine-1- | | | |
| | carboxylate | | | |
| 18 | (4-(4-(5-((2- | 473.91 | 99.2 | (4) |
| | chlorobenzyl)oxy)pyridin-3-yl)-1H- | | | |
| | pyrazol-1-yl)piperidin-1- | | | |
| | yl)(phenyl)methanone | | | |
| 19 | Benzyl 4-(4-(5-((2- | 504.47 | 27.8 | (4) |
| | chlorobenzyl)oxy)pyridin-3-yl)-1H- | | | |
| | pyrazol-1-yl)piperidine-1- | | | |
| | carboxylate | | | |
| 20 | 3-((3-chlorobenzyl)oxy)-5-(1- | 369.35 | NT | |
| | (piperidin-4-yl)-1H-pyrazol-4- | | | |
| | yl)pyridine | | | |
| 21 | (S)-3-(1-(2-chlorophenyl)ethoxy)-5- | 383.30 | 344 | (3) |
| | (1-(piperidin-4-yl)-1H-pyrazol-4- | | | |
| | yl)pyridine | | | |
| 22 | (R)-3-(1-(2-chlorophenyl)ethoxy)-5- | 383.30 | 152 | (1) |
| | (1-(piperidin-4-yl)-1H-pyrazol-4- | | | |
| | yl)pyridine | | | |
| 23 | (R)-3-(1-(4-chlorophenyl)ethoxy)-5- | 383.24 | 228 | (3) |
| | (1-(piperidin-4-yl)-1H-pyrazol-4- | | | |
| | yl)pyridine | | | |
| 24 | (S)-3-(1-(4-chlorophenyl)ethoxy)-5- | 383.30 | NT | |
| | (1-(piperidin-4-yl)-1H-pyrazol-4- | | | |
| | yl)pyridine | | | |
| 25 | 3-[(2-Methylphenyl)methoxy]-5-[1- | 349.37 | 851 | (3) |
| | (piperidin-4-yl)-1H-pyrazol-4- | | | |
| | yl]pyridine | | | |
| 26 | 3-((2-chlorobenzyl)oxy)-5-(1- | 369.25 | 690 | (3) |
| | (piperidin-4-yl)-1H-pyrazol-3- | | | |
| | yl)pyridine | | | |
| 27 | 3-((2-chlorobenzyl)oxy)-5-(3- | 369.38 | 332 | (1) |
| | (piperidin-4-yl)-1H-pyrazol-1- | | | |
| | yl)pyridine | | | |
| 28 | 3-((2-chlorobenzyl)oxy)-5-(4- | 369.38 | 601 | (2) |
| | (piperidin-4-yl)-1H-pyrazol-1- | | | |
| | yl)pyridine | | | |
| 29 | 4-(4-(3-((2- | 368.30 | 186 | (3) |
| | chlorobenzyl)oxy)phenyl)-1H- | | | |
| | pyrazol-1-yl)piperidine | | | |

TABLE 2

| Compound | Name | MS (ES+) | SHIP1 IC₅₀ μM (n) |
|---|---|---|---|
| 1 | 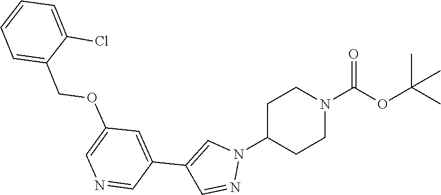 [SMILES: CC(C)(C)OC(=O)N1CCC(n2cc(-c3cncc(OCc4ccccc4Cl)c3)cn2)CC1] | 469.22 | 92.1 (3) |
| 2 | 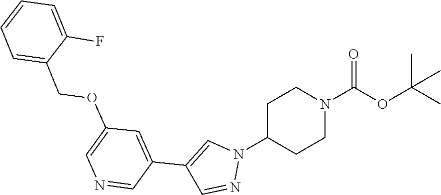 [SMILES: CC(C)(C)OC(=O)N1CCC(n2cc(-c3cncc(OCc4ccccc4F)c3)cn2)CC1] | 453.38 | 81.9 (8) |
| 3 | 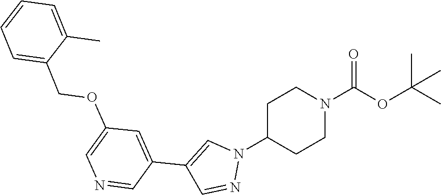 [SMILES: Cc1ccccc1COc1cncc(-c2cnn(C3CCN(C(=O)OC(C)(C)C)CC3)c2)c1] | 449.38 | 116 (4) |
| 4 | 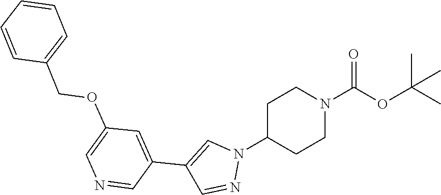 [SMILES: CC(C)(C)OC(=O)N1CCC(n2cc(-c3cncc(OCc4ccccc4)c3)cn2)CC1] | 435.40 | 90.3 (4) |
| 5 | 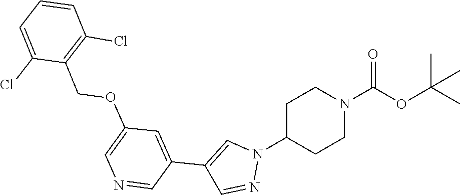 [SMILES: CC(C)(C)OC(=O)N1CCC(n2cc(-c3cncc(OCc4c(Cl)cccc4Cl)c3)cn2)CC1] | 503.30 | 35.7 (3) |
| 6 | 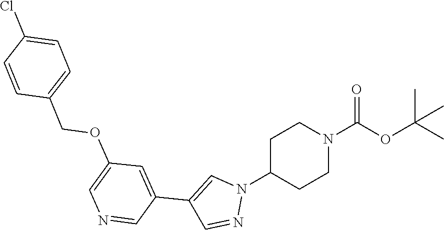 [SMILES: CC(C)(C)OC(=O)N1CCC(n2cc(-c3cncc(OCc4ccc(Cl)cc4)c3)cn2)CC1] | 469.22 | 137 (3) |
| 7 | 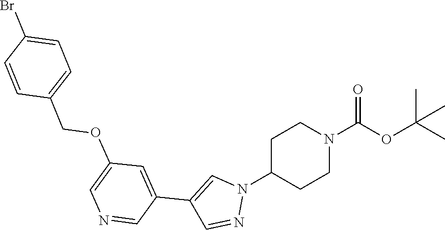 [SMILES: CC(C)(C)OC(=O)N1CCC(n2cc(-c3cncc(OCc4ccc(Br)cc4)c3)cn2)CC1] | 515.22 | 57.0 (4) |
| 8 | 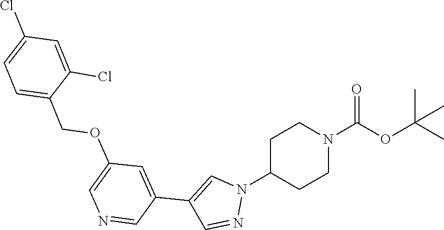 [SMILES: CC(C)(C)OC(=O)N1CCC(n2cc(-c3cncc(OCc4ccc(Cl)cc4Cl)c3)cn2)CC1] | 503.30 | 18.1 (1) |
| 9 | 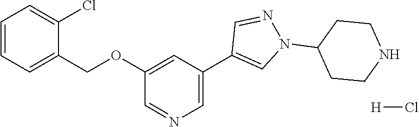 | 369.25 | 302 (8) |
| 10 | 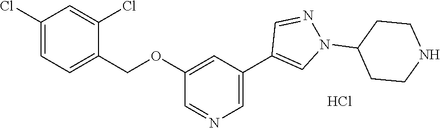 [SMILES: Clc1ccc(COc2cncc(-c3cnn(C4CCNCC4)c3)c2)c(Cl)c1 ; Cl] | 403.10 | 286 (9) |
| 11 | 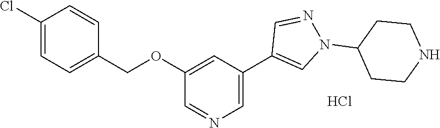 [SMILES: Clc1ccc(COc2cncc(-c3cnn(C4CCNCC4)c3)c2)cc1 ; Cl] | 369.16 | 109 (3) |
| 12 | 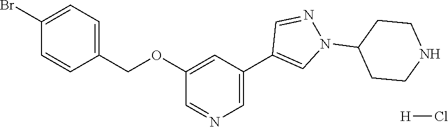 | 413.08 | 29.7 (3) |
| 13 | 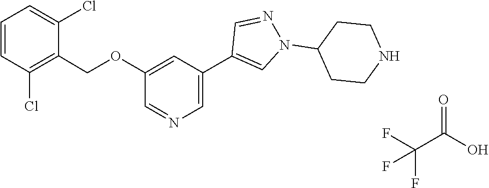 [SMILES: Clc1cccc(Cl)c1COc1cncc(-c2cnn(C3CCNCC3)c2)c1 ; O=C(O)C(F)(F)F] | 403.20 | 147 (4) |
| 14 | 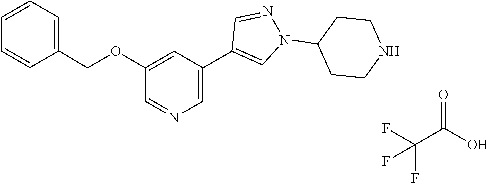 [SMILES: c1ccc(COc2cncc(-c3cnn(C4CCNCC4)c3)c2)cc1 ; O=C(O)C(F)(F)F] | 334.18 | 208 (4) |
| 15 | 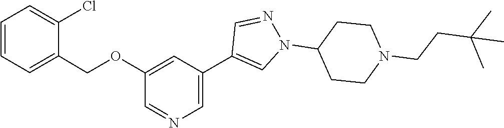 [SMILES: CC(C)(C)CCN1CCC(n2cc(-c3cncc(OCc4ccccc4Cl)c3)cn2)CC1] | 453.35 | 43.3 (4) |
| 16 | 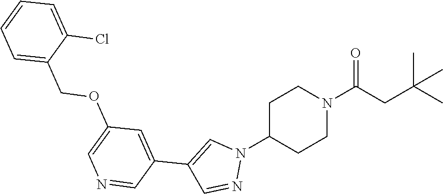 [SMILES: CC(C)(C)CC(=O)N1CCC(n2cc(-c3cncc(OCc4ccccc4Cl)c3)cn2)CC1] | 467.37 | 102 (4) |
| 17 | 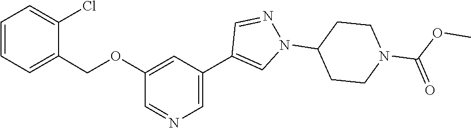 [SMILES: COC(=O)N1CCC(n2cc(-c3cncc(OCc4ccccc4Cl)c3)cn2)CC1] | 427.30 | 133 (3) |
| 18 | 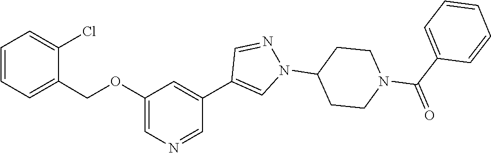 [SMILES: O=C(c1ccccc1)N1CCC(n2cc(-c3cncc(OCc4ccccc4Cl)c3)cn2)CC1] | 473.91 | 99.2 (4) |
| 19 | 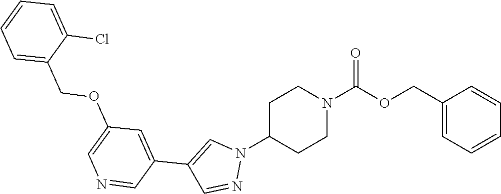 [SMILES: O=C(OCc1ccccc1)N1CCC(n2cc(-c3cncc(OCc4ccccc4Cl)c3)cn2)CC1] | 504.47 | 27.8 (4) |
| 20 | 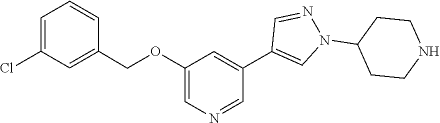 [SMILES: Clc1cccc(COc2cncc(-c3cnn(C4CCNCC4)c3)c2)c1] | 369.35 | NT |
| 21 | 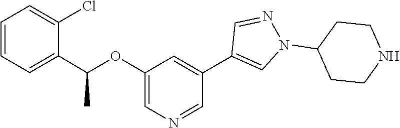 [SMILES: C[C@H](Oc1cncc(-c2cnn(C3CCNCC3)c2)c1)c1ccccc1Cl] | 383.30 | 344 (3) |
| 22 | 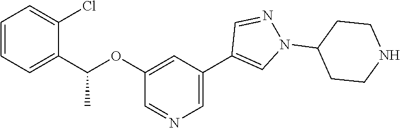 [SMILES: C[C@@H](Oc1cncc(-c2cnn(C3CCNCC3)c2)c1)c1ccccc1Cl] | 383.30 | 152 (1) |
| 23 | 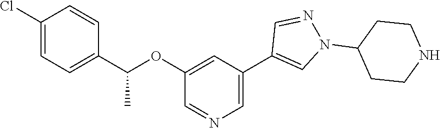 [SMILES: C[C@@H](Oc1cncc(-c2cnn(C3CCNCC3)c2)c1)c1ccc(Cl)cc1] | 383.24 | 228 (3) |
| 24 | 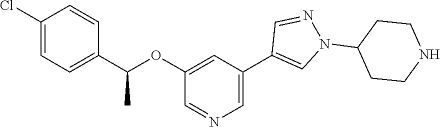 | 383.30 | NT |
| 25 | 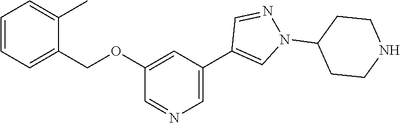 | 349.37 | 851 (3) |
| 26 | 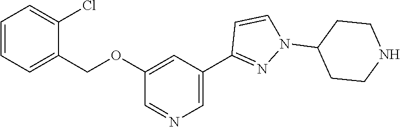 | 369.25 | 690 (3) |
| 27 | 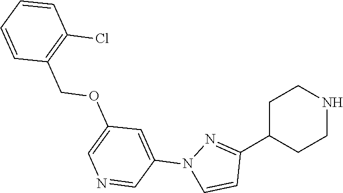 [SMILES: Clc1ccccc1COc1cncc(-n2ccc(C3CCNCC3)n2)c1] | 369.38 | 332 (1) |
| 28 | 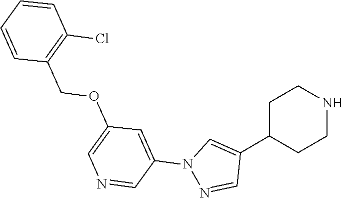 [SMILES: Clc1ccccc1COc1cncc(-n2cc(C3CCNCC3)cn2)c1] | 369.38 | 601 (2) |
| 29 | 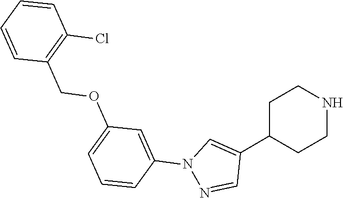 [SMILES: Clc1ccccc1COc1cccc(-n2cc(C3CCNCC3)cn2)c1] | 368.30 | 186 (3) |

### Example 1

### Intermediate 1

### tert-butyl 4-(4-(5-hydroxypyridin-3-yl)-1H-pyrazol-1-yl)piperidine-1-carboxylate

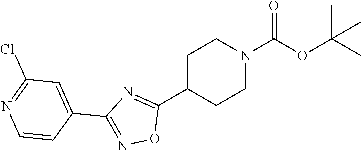 [SMILES: CC(C)(C)OC(=O)N1CCC(c2nc(-c3ccnc(Cl)c3)no2)CC1]

To a stirred solution of 5-bromopyridin-3-ol (1 g, 5.75 mmol) and tert-butyl 4-[4-(4,4,5,5-tetramethyl-1,3,2-dioxaborolan-2-yl)-1H-pyrazol-1-yl]piperidine-1-carboxylate (2.17 g, 5.75 mmol) in toluene (5 mL, 42.3 mmol), ethanol (5 mL, 85.6 mmol) and water (2 mL, 111 mmol), was added dipotassium carbonate (1.59 g, 2 eq., 11.5 mmol) and reaction mixture was purged with nitrogen for 20 minutes. Then [1,1′-Bis(diphenylphosphino)ferrocene]dichloropalladium(II) (234 mg, 0.05 eq., 287 μmol) was added and reaction mixture was heated at 100° C. and stirred for 16 h. Reaction mixture was monitored by TLC and LCMS. Reaction mixture was diluted with water, extracted with EtOAc, washed with brine, dried over anhydrous sodium sulfate, filtered and concentrated. Crude was purified by silica gel flash chromatography using 40% EtOAc in hexane as an eluent, desired fractions were concentrated to afford tert-butyl 4-[4-(5-hydroxypyridin-3-yl)-1H-pyrazol-1-yl]piperidine-1-carboxylate (0.5 g, 1.39 mmol, 24.2%) as a white solid. MS 343.12.

### Example 2

### Compound 1

### tert-butyl 4-(4-(5-((2-chlorobenzyl)oxy)pyridin-3-yl)-1H-pyrazol-1-yl)piperidine-1-carboxylate

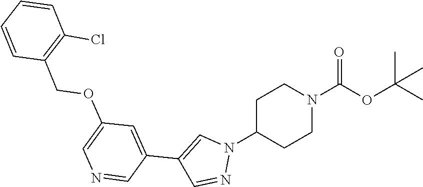 [SMILES: CC(C)(C)OC(=O)N1CCC(n2cc(-c3cncc(OCc4ccccc4Cl)c3)cn2)CC1]

To a stirred solution of tert-butyl 4-[4-(5-hydroxypyridin-3-yl)-1H-pyrazol-1-yl]piperidine-1-carboxylate (0.4 g, 1.16 mmol) and 1-(bromomethyl)-2-chlorobenzene (263 mg, 1.1 eq., 1.28 mmol) in dimethylformamide (7 mL, 90.4 mmol), was added dipotassium carbonate (321 mg, 2 eq., 2.32 mmol) portion wise and reaction mixture heated at 100° C. for 16 h. After completion (TLC and LCMS monitoring), reaction mixture was diluted with water, extracted with EtOAc, washed with brine, dried over anhydrous sodium sulfate, filtered and concentrated. Crude was purified by silica gel flash chromatography using 40% EtOAc in hexane as an eluent, desired fractions were concentrated to afford tert-butyl 4-(4-(5-((2-chlorophenyl)methoxy)pyridin-3-yl)-1H-pyrazol-1-yl)piperidine-1-carboxylate (180 mg, 365 μmol, 31.4%) as an off white solid. MS 469.22.

Compounds 2-8 were all prepared in a similar manner to Compound 1.

| Com- | | |
|---|---|---|
| pound | | MS |
| # | Name | (ES+) |
| 2 | tert-butyl 4-(4-(5-((2-fluorobenzyl)oxy)pyridin-3-yl)- | 453.38 |
| | 1H-pyrazol-1-yl)piperidine-1-carboxylate | |
| 3 | tert-butyl 4-(4-(5-((2-methylbenzyl)oxy)pyridin-3-yl)- | 449.38 |
| | 1H-pyrazol-1-yl)piperidine-1-carboxylate | |
| 4 | tert-butyl 4-(4-(5-(benzyloxy)pyridin-3-yl)-1H-pyrazol- | 435.40 |
| | 1-yl)piperidine-1-carboxylate | |
| 5 | tert-butyl 4-(4-(5-((2,6-dichlorobenzyl)oxy)pyridin-3- | 503.30 |
| | yl)-1H-pyrazol-1-yl)piperidine-1-carboxylate | |
| 6 | tert-butyl 4-(4-(5-((4-chlorobenzyl)oxy)pyridin-3-yl)- | 469.22 |
| | 1H-pyrazol-1-yl)piperidine-1-carboxylate | |
| 7 | tert-butyl 4-(4-(5-((4-bromobenzyl)oxy)pyridin-3-yl)- | 515.22 |
| | 1H-pyrazol-1-yl)piperidine-1-carboxylate | |
| 8 | tert-butyl 4-(4-(5-((2,4-dichlorobenzyl)oxy)pyridin-3- | 503.30 |
| | yl)-1H-pyrazol-1-yl)piperidine-1-carboxylate | |

### Example 3

### Compound 9

### 3-((2-chlorobenzyl)oxy)-5-(1-(piperidin-4-yl)-1H-pyrazol-4-yl)pyridine. HCl

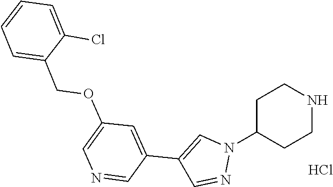 [SMILES: Clc1ccccc1COc1cncc(-c2cnn(C3CCNCC3)c2)c1 ; Cl]

To a stirred solution of tert-butyl 4-(4-(5-[(2-chlorophenyl)methoxy]pyridin-3-yl)-1H-pyrazol-1-yl)piperidine-1-carboxylate (180 mg, 384 μmol) in dichloromethane (5 mL, 78.1 mmol), was added 2 mL of 4M HCl in dioxane drop wise at 0° C. and reaction mixture stirred at room temperature for 3 h. After completion (TLC and LCMS monitoring), reaction mixture was concentrated and titurated with n-pentane and diethyl ether to afford 3-((2-chlorobenzyl)oxy)-5-(1-(piperidin-4-yl)-1H-pyrazol-4-yl)pyridine hydrochloride salt (91 mg, 222 μmol, 57.9%) as an off white solid. MS 369.25.

### Example 4

### Compound 10

### 3-((2,4-dichlorobenzyl)oxy)-5-(1-(piperidin-4-yl)-1H-pyrazol-4-yl)pyridine. HCl

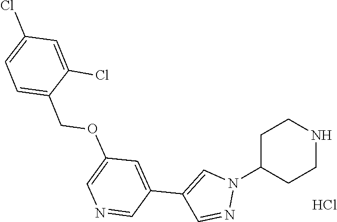 [SMILES: Clc1ccc(COc2cncc(-c3cnn(C4CCNCC4)c3)c2)c(Cl)c1 ; Cl]

To a stirred solution of tert-butyl 4-(4-(5-[(2,4-dichlorophenyl)methoxy]pyridin-3-yl)-1H-pyrazol-1-yl)piperidine-1-carboxylate (250 mg, 497 μmol) in dichloromethane (5 mL, 78.1 mmol), was added 2 mL of 4M HCl in dioxane drop wise at 0° C. and reaction mixture stirred at room temperature for 3 h. After completion (TLC and LCMS monitoring), reaction mixture was concentrated and titurated with n-pentane and diethyl ether to afford 3-[(2,4-dichlorophenyl)methoxy]-5-[1-(piperidin-4-yl)-1H-pyrazol-4-yl]pyridine hydrochloride salt (170 mg, 422 μmol, 85%) as an off white solid. MS 403.10.

### Compounds 11 and 12 were Prepared in a Similar Manner to Compound 10

| | | MS |
|---|---|---|
| Compound | Name | (ES+) |
| 11 | 3-((4-chlorobenzyl)oxy)-5-(1-(piperidin-4-yl)-1H- | 369.16 |
| | pyrazol-4-yl)pyridine hydrochoride | |
| 12 | 3-((4-bromobenzyl)oxy)-5-(1-(piperidin-4-yl)-1H- | 413.08 |
| | pyrazol-4-yl)pyridine hydrochoride | |

### Example 5

### Compound 13

### 3-((2,6-dichlorobenzyl)oxy)-5-(1-(piperidin-4-yl)-1H-pyrazol-4-yl)pyridine trifluoroacetate

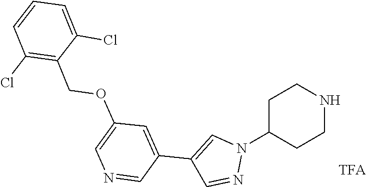 [SMILES: Clc1cccc(Cl)c1COc1cncc(-c2cnn(C3CCNCC3)c2)c1 ; "TFA"]

To a stirred solution of tert-butyl 4-(4-(5-((2,6-dichlorobenzyl)oxy)pyridin-3-yl)-1H-pyrazol-1-yl)piperidine-1-carboxylate (225 mg, 447 μmol) in dichloromethane (5 mL, 78.1 mmol), was added trifluoroacetic acid (1 mL) drop wise at 0° C. and reaction mixture stirred at room temperature for 3 h. After completion (TLC and LCMS monitoring), reaction mixture was concentrated and titurated with n-pentane and diethyl ether. The compound was purified by prep HPLC using 1% aqueous TFA/acetonitrile to afford to afford 3-((2,6-dichlorobenzyl)oxy)-5-(1-(piperidin-4-yl)-1H-pyrazol-4-yl)pyridine (18 mg, 42.8 μmol, 9.6%) as an off white trifluoroacetate salt. MS 403.20.

Compound 14 was prepared in a similar manner to Compound 10

| | | MS |
|---|---|---|
| Compound | Name | (ES+) |
| 14 | 3-(benzyloxy)-5-(1-(piperidin-4-yl)-1H-pyrazol-4- | 335.35 |
| | yl)pyridine | |

### Example 6

### Compound 15

### 3-((2-chlorobenzyl)oxy)-5-(1-(1-(3,3-dimethylbutyl)piperidin-4-yl)-1H-pyrazol-4-yl)pyridine

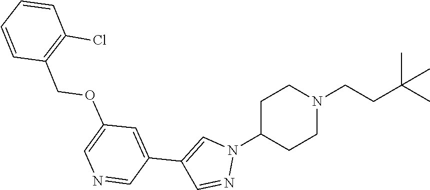 [SMILES: CC(C)(C)CCN1CCC(n2cc(-c3cncc(OCc4ccccc4Cl)c3)cn2)CC1]

To a stirred solution of 3-((2-chlorophenyl)methoxy)-5-(1-(piperidin-4-yl)-1H-pyrazol-4-yl)pyridine (150 mg, 407 μmol) and 1-bromo-3,3-dimethylbutane (67.1 mg, 407 μmol) in dimethylformamide (3 mL, 38.7 mmol), was added dipotassium carbonate (169 mg, 3 eq., 1.22 mmol) portion wise and reaction mixture heated at 100° C. for 16 h. After completion (TLC and LCMS monitoring), reaction mixture was diluted with water, extracted with EtOAc, washed with brine, dried over anhydrous sodium sulfate, filtered and concentrated. Crude was purified by silica gel flash chromatography using 40% EtOAc in hexane as an eluent, desired fractions were concentrated to afford 3-((2-chlorobenzyl)oxy)-5-(1-(1-(3,3-dimethylbutyl)piperidin-4-yl)-1H-pyrazol-4-yl)pyridine (15 mg, 33.1 μmol, 8.1%) as an off white solid. MS 453.35.

### Example 7

### Compound 16

### 1-(4-(4-(5-((2-chlorobenzyl)oxy)pyridin-3-yl)-1H-pyrazol-1-yl)piperidin-1-yl)-3,3-dimethylbutan-1-one

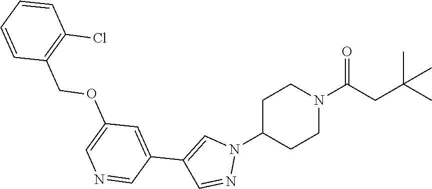 [SMILES: CC(C)(C)CC(=O)N1CCC(n2cc(-c3cncc(OCc4ccccc4Cl)c3)cn2)CC1]

To the stirred solution of 3-((2-chlorophenyl)methoxy)-5-(1-(piperidin-4-yl)-1H-pyrazol-4-yl)pyridine (0.1 g, 271 μmol) in dichloromethane (2 mL, 31.2 mmol) was added triethylamine (114 μL, 3 eq., 813 μmol) followed by 3,3-dimethylbutanoyl chloride (54.7 mg, 1.5 eq., 407 μmol) at 0° C. Reaction mixture was stirred at room temperature for 16 h. After completion (TLC & LCMS monitoring), reaction mixture was diluted with water, extracted with DCM. Organic layer was washed with brine, dried over anhydrous sodium sulfate, concentrated. Crude was purified using 10% MeOH in DCM as an eluent, desired fractions were concentrated to afford 1-(4-(4-(5-((2-chlorobenzyl)oxy)pyridin-3-yl)-1H-pyrazol-1-yl)piperidin-1-yl)-3,3-dimethylbutan-1-one (38 mg, 79.2 μmol, 29.2%) as yellowish semi solid. MS 467.37.

### Example 8

### Compound 17

### Methyl 4-(4-(5-((2-chlorobenzyl)oxy)pyridin-3-yl)-1H-pyrazol-1-yl)piperidine-1-carboxylate

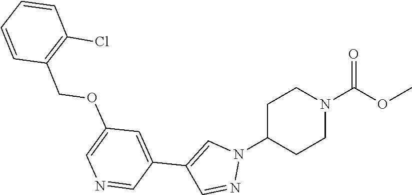 [SMILES: COC(=O)N1CCC(n2cc(-c3cncc(OCc4ccccc4Cl)c3)cn2)CC1]

To the stirred solution of 3-[(2-chlorophenyl)methoxy]-5-[1-(piperidin-4-yl)-1H-pyrazol-4-yl]pyridine (150 mg, 407 μmol) in dichloromethane (3 mL, 46.9 mmol) was added triethylamine (170 μL, 3 eq., 1.22 mmol) followed by methyl chloroformate (57.6 mg, 1.5 eq., 610 μmol) at 0° C. Reaction mixture was stirred at room temperature for 16 h. After completion (TLC & LCMS monitoring), reaction mixture was diluted with water, extracted with DCM. Organic layer was washed with brine, dried over anhydrous sodium sulfate, concentrated. The crude material was purified using 10% MeOH in DCM as an eluent, desired fractions were concentrated to afford methyl 4-(4-(5-((2-chlorobenzyl)oxy)pyridin-3-yl)-1H-pyrazol-1-yl)piperidine-1-carboxylate (53 mg, 118 μmol, 29.0%) as yellowish semi solid. MS 427.30.

### Example 9

### Compound 18

### (4-(4-(5-((2-chlorobenzyl)oxy)pyridin-3-yl)-1H-pyrazol-1-yl)piperidin-1-yl)(phenyl)methanone

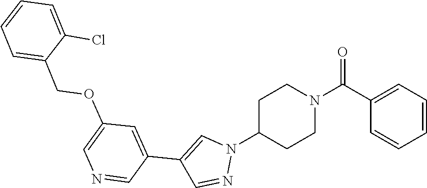 [SMILES: O=C(c1ccccc1)N1CCC(n2cc(-c3cncc(OCc4ccccc4Cl)c3)cn2)CC1]

To a stirred solution of 3-((2-chlorobenzyl)oxy)-5-(1-(piperidin-4-yl)-1H-pyrazol-4-yl)pyridine hydrochloride salt (75 mg, 170 μmol) in tetrahydrofuran (2 mL, 24.6 mmol) was added benzoyl chloride (24 μL, 1.2 eq., 207 μmol) and N,N-diisopropylethylamine (125 μL, 4.2 eq., 718 μmol) at room temperature. The reaction mixture was stirred for 2 hours at which time LCMS indicated the reaction was complete. The reaction mixture was diluted with the water (2 mL) and extracted with ethyl acetate (2 mL). The organic layer was dried over anhydrous sodium sulfate, filtered and concentrated under reduced pressure to obtain the crude compound. The crude material was purified by silica gel flash chromatography using 0-20% MeOH in dichloromethane to afford the desired product (4-(4-(5-((2-chlorobenzyl)oxy)pyridin-3-yl)-1H-pyrazol-1-yl)piperidin-1-yl)(phenyl)methanone (62 mg, 131 μmol, 77%) as a yellow oil. MS 473.91.

### Example 10

### Compound 19

### Benzyl 4-(4-(5-((2-chlorobenzyl)oxy)pyridin-3-yl)-1H-pyrazol-1-yl)piperidine-1-carboxylate

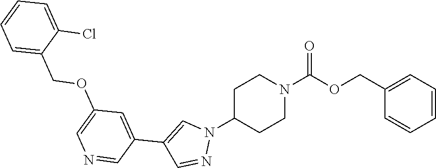 [SMILES: O=C(OCc1ccccc1)N1CCC(n2cc(-c3cncc(OCc4ccccc4Cl)c3)cn2)CC1]

To a stirred solution of 3-((2-chlorobenzyl)oxy)-5-(1-(piperidin-4-yl)-1H-pyrazol-4-yl)pyridine hydrochloride salt (75 mg, 170 μmol) in tetrahydrofuran (2 mL, 24.6 mmol) was added benzyl chloroformate (29 μL, 1.2 eq., 204 μmol) and N,N-diisopropylethylamine (149 μL, 5 eq., 856 μmol) at room temperature. The reaction mixture was stirred for 1.5 hours at which time LCMS indicated the reaction was complete. The reaction mixture was diluted with the water (2 mL) and extracted with ethyl acetate (3×2 mL). The combined organic layers were dried over anhydrous sodium sulfate, filtered and concentrated under reduced pressure to obtain the crude compound. The crude material was purified by silica gel flash chromatography using 0-85% EtOAc in n-hexanes to afford the desired product benzyl 4-(4-(5-((2-chlorobenzyl)oxy)pyridin-3-yl)-1H-pyrazol-1-yl)piperidine-1-carboxylate (32 mg, 63.6 μmol, 37%) as a yellow oil. MS 504.47.

### Example 11

### Compound 20

### 3-((3-Chlorobenzyl)oxy)-5-(1-(piperidin-4-yl)-1H-pyrazol-4-yl)pyridine

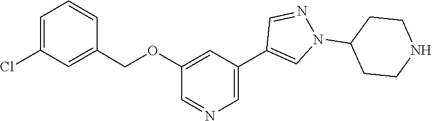 [SMILES: Clc1cccc(COc2cncc(-c3cnn(C4CCNCC4)c3)c2)c1]

### Intermediate 1 (starting material for Intermediate 2, which is the starting material for Compound 20) (3-bromo-5-((3-chlorobenzyl)oxy)pyridine)

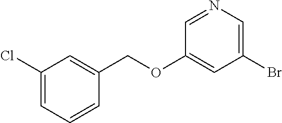 [SMILES: Clc1cccc(COc2cncc(Br)c2)c1]

To a stirred solution of cesium carbonate (206 mg, 1.1 eq., 632 μmol) and 5-bromopyridin-3-ol (100 mg, 575 μmol) in dimethylformamide (12 mL, 155 mmol) was added 1-(bromomethyl)2-chlorobenzene (118 mg, 1 eq., 575 μmol). The reaction mixture was heated and stirred in an Anton Paar Monowave 450 microwave at 70° C. for 2 hours. After completion the solution was diluted with ethyl acetate and washed five times with brine. Reaction mixture was concentrated. Crude was purified by flash chromatography using 60% ethyl acetate in hexane as an eluent. Desired fractions were concentrated to afford 3-bromo-5-((3-chlorebenzyl)oxy)pyridine (163 mg, 544 μmol). MS 296.96.

### Intermediate 2 (Starting Material for Compound 20) (tert-butyl 4-(4-(5-((3-chlorobenzyl)oxy)pyridin-3-yl)-1H-pyrazol-1-yl)piperidine-1-carboxylate)

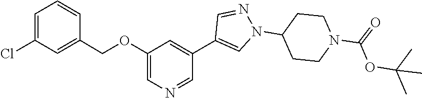 [SMILES: CC(C)(C)OC(=O)N1CCC(n2cc(-c3cncc(OCc4cccc(Cl)c4)c3)cn2)CC1]

To a reaction vessel was added 3-bromo-5-((3-chlorobenzyl)oxy)pyridine (170 mg, 0.98 eq., 569 μmol), tert-butyl 4-[4,5,6,6-tetramethyl-1,3,2-dioxaborolan-2-yl)-1H-pyrazol-1-yl]piperidine-1-carboxylate (262 mg, 1.1 eq. 640 μmol), 1,4-dioxane (5.82 mL, 68.3 mmol), dipotassium carbonate (262 mg, 3.3 eq., 1.89 mmol), and water (1.11 mL, 61.7 mmol). The reaction mixture was frozen, vacuumed, purged with nitrogen, and thawed. This cycle was carried out three times. Pd(dppf)Cl₂ (21.6 mg, 0.05 eq., 29.1 μmol) was added and the mixture was heated to 90° C. for 4 h. After the reaction was complete (monitored by LCMS) the mixture was poured into water and extracted with ethyl acetate. The organic layer was washed with water and brine. The organics were dried with Na₂SO₄, filtered, and concentrated to crude material. Crude was purified by flash chromatography using 0-100% EtOAc in hexane gradient as an eluent, desired fractions were concentrated to afford tert-butyl 4-(4-(5-((3-chlorobenzyl)oxy)pyridin-3-yl)-1H-pyrazol-1-yl)piperidine-1-carboxylate as an off white solid (126 mg, 269 μmol). MS (M+AcCN) 510.45.

### 3-((3-chlorobenzyl)oxy)-5-(1-(piperidin-4-yl)-1H-pyrazol-4-yl)pyridine (Compound 20)

To a stirred solution of tert-butyl 4-(4-(5-((3-chlorobenzyl)oxy)pyridin-3-yl)-1H-pyrazol-1-yl)piperidine-1-carboxylate (126 mg, 269 μmol) in dichloromethane (2 mL, 31.4 mmol) was added 4 N hydrochloric acid in dioxane (0.5 mL, 2 mmol) at room temperature. After 2 hours additional 4 N hydrochloric acid in dioxane (0.5 mL, 2 mmol) was added and the reaction mixture was stirred overnight. After completion (monitored by LCMS) the solution was concentrated in vacuo and washed with methyl tert-butyl ether (2×2 mL). The remaining solids were dried in vacuo to afford the HCl salt of 3-((3-chlorobenzyl)oxy)-5-(1-(piperidin-4-yl)-1H-pyrazol-4-yl)pyridine as a white solid. MS 369.35.

### Example 12

### Compound 21

### (S)-3-(1-(2-chlorophenyl)ethoxy)-5-(1-(piperidin-4-yl)-1H-pyrazol-4-yl)pyridine

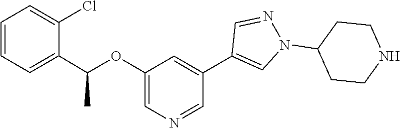 [SMILES: C[C@H](Oc1cncc(-c2cnn(C3CCNCC3)c2)c1)c1ccccc1Cl]

### Intermediate 3 (Starting Material for Intermediate 4, which is the Starting Material for Compound 21) (tert-butyl 4-(4-(5-hydroxypyridin-3-yl)-1H-pyrazol-1-yl)piperidine-1-carboxylate)

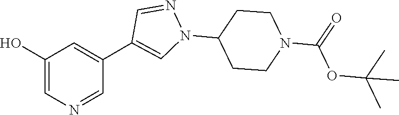 [SMILES: CC(C)(C)OC(=O)N1CCC(n2cc(-c3cncc(O)c3)cn2)CC1]

To a stirred solution of 5-bromopyridin-3-ol (2.01 g, 11.6 mmol) and tert-butyl 4-[4-(4,4,5,5-tetramethyl-1,3,2-dioxaborolan-2-yl)-1H-pyrazol-1-yl)piperidine-1-carboxylate (4.36 g, 11.6 mmol) in toluene (16 mL, 151 mmol), was added dipotassium carbonate (3.2 g, 2 eq., 23.1 mmol). Reaction mixture was purged with argon for 20 min. Then bis(diphenylphosphino)ferrocene palladium dichloride (472 mg, 0.05 eq., 578 μmol) was added and reaction mixture was heated to 100° C. and stirred for 16 h. Reaction mixture was monitored by TLC and LCMS. Reaction mixture was concentrated. Crude was purified by flash chromatography using 60% ethyl acetate in hexane as an eluent. Desired fractions were concentrated to afford tert-butyl 4-[4-(5-hydroxypyridin-3-yl)-1H-pyrazol-1-yl]piperidine-1-carboxylate (1.2 g, 3.31 mmol) as an off-white solid. MS 345.25.

### Intermediate 4 (Starting Material for Compound 21) (tert-butyl (S)-4-(4-(5-(1-(2-chlorophenyl)ethoxy)pyridin-3-yl)-1H-pyrazol-1-yl)piperidine-1-carboxylate)

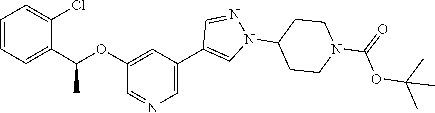 [SMILES: C[C@H](Oc1cncc(-c2cnn(C3CCN(C(=O)OC(C)(C)C)CC3)c2)c1)c1ccccc1Cl]

To a stirred solution of tert-butyl 4-(4-(5-hydroxypyridin-3-yl)-1H-pyrazol-1-yl)piperidine-1-carboxylate (0.5 g. 1.45 mmol) and (1R)-1-(2-clorophenyl)ethan-1-ol (238 mg, 1.52 mmol) in tetrahydrofuran (20 mL, 246 mmol), was added triphenylphosphane (799 mg, 2 eq., 3.04 mmol) portion wise and reaction mixture stirred at room temperature for 1 h. Then (E)-N-[(ethoxycarbonyl)imino]ethoxyformamide (530 mg, 2 eq., 3.04 mmol) was added in stock solution of tetrahydrofuran dropwise and reaction mixture stirred at room temperature for 16 h. After completion (TLC and LCMS monitoring). Reaction mixture was diluted with water, extracted with EtOAc, washed with brine, dried over anhydrous sodium sulfate, filtered and concentrated. Crude was purified by flash chromatography using 70% EtOAc in hexane as an eluent, desired fractions were concentrated to afford tert-butyl (S)-4-(4-(5-(1-(2-chlorophenyl)ethoxy)pyridin-3-yl)-1H-pyrazol-1-yl)piperidine-1-carboxylate (180 mg, 373 μmol) as a sticky brown. MS 482.90.

### (S)-3-(1-(2-chlorophenyl)ethoxy)-5-(1-(piperidin-4-yl)-1H-pyrazol-4-yl)pyridine (Compound 21)

To a stirred solution of tert-butyl (S)-4-(4-(5-(1-(2-chlorophenyl)ethoxy)pyridin-3-yl)-1H-pyrazol-1-yl)piperidine-1-carboxylate (180 mg, 373 μmol) in dichloromethane (4 mL, 62.5 mmol), was added trifluoracetic acid (1 mL) drop wise at 0° C. and reaction mixture stirred at room temperature for 3 h. After completion (TLC and LCMS monitoring), reaction mixture was concentrated and triturated with n-pentane and diethyl ether. Submitted to prep HPLC using TFA buffer to afford the TFA salt of (S)-3-(1-(2-chlorophenyl)ethoxy)-5-(1-(piperidin-4-yl)-1H-pyrazol-4-yl)pyridine (45 mg, 89.7 μmol) as an off white solid. MS 383.30.

### Example 13

### Compound 22

### (R)-3-(1-(2-chlorophenyl)ethoxy)-5-(1-(piperidin-4-yl)-1H-pyrazol-4-yl)pyridine

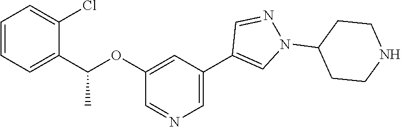 [SMILES: C[C@@H](Oc1cncc(-c2cnn(C3CCNCC3)c2)c1)c1ccccc1Cl]

Prepared in a manner substantially similar to (S)-3-(1-(2-chlorophenyl)ethoxy)-5-(1-(piperidin-4-yl)-1H-pyrazol-4-yl)pyridine. MS 383.30.

### Example 14

### Compound 23

### (R)-3-(1-(4-chlorophenyl)ethoxy)-5-(1-(piperidin-4-yl)-1H-pyrazol-4-yl)pyridine

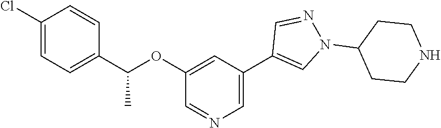 [SMILES: C[C@@H](Oc1cncc(-c2cnn(C3CCNCC3)c2)c1)c1ccc(Cl)cc1]

### Intermediate 5 (Starting Material for Compound 23) (tert-butyl (R)-4-(4-(5-(1-(4-chlorophenyl)ethoxy)pyridin-3-yl)-1H-pyrazol-1-yl)piperidine-1-carboxylate)

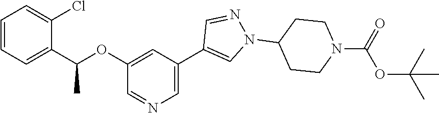 [SMILES: C[C@H](Oc1cncc(-c2cnn(C3CCN(C(=O)OC(C)(C)C)CC3)c2)c1)c1ccccc1Cl]

To a stirred solution of tert-butyl-4-(4-(5-hydroxy-3-pyridyl)-1-pyrazolyl)-1-piperidine carboxylate (440 mg. 1.28 mmol) in dimethylformamide (12.8 mL, 165 mmol), was added dipotassium carbonate (353 mg, 2 eq., 2.56 mmol) and reaction mixture was stirred for 5 min. Then (S)-1-(p-chlorophenyl)-1-(mesyloxy)ethane (0.3 g, 1.28 mmol) was added and reaction mixture was heated at 70° C. and stirred for 12 h. Reaction mixture was diluted with water, extracted with EtOAc, washed with brine, dried over anhydrous sodium sulfate, filtered and concentrated. Crude was purified by flash chromatography using 0-100% EtOAc in hexane gradient as an eluent, desired fractions were concentrated to afford tert-butyl (R)-4-(4-(5-(1-(4-chlorophenyl)ethoxy)pyridin-3-yl)-1H-pyrazol-1-yl)piperidine-1-carboxylate (129.24 mg, 1 μmol) as a yellow oil. MS (M+AcCN) 524.50.

### (R)-3-(1-(4-chlorophenyl)ethoxy)-5-(1-(piperidin-4-yl)-1H-pyrazol-4-yl)pyridine (Compound 23)

To a stirred solution of tert-butyl (R)-4-(4-(5-(1-(4-chlorophenyl)ethoxy)pyridin-3-yl)-1H-pyrazol-1-yl)piperidine-1-carboxylate (130 mg, 269 μmol) in dichloromethane (2 mL, 31.4 mmol) was added 4 N hydrochloric acid in dioxane (0.5 mL, 2 mmol) at room temperature. After 2 hours additional 4 N hydrochloric acid in dioxane (0.5 mL, 2 mmol) was added and the reaction mixture was stirred overnight. After completion (monitored by LCMS) the solution was concentrated in vacuo and washed with methyl tert-butyl ether (2×2 mL). The remaining solids were dried in vacuo to afford the HCl salt of (R)-3-(1-(4-chlorophenyl)ethoxy)-5-(1-(piperidin-4-yl)-1H-pyrazol-4-yl)pyridine. MS 383.24.

### Example 15

### Compound 24

### (S)-3-(1-(4-chlorophenyl)ethoxy)-5-(1-(piperidin-4-yl)-1H-pyrazol-4-yl)pyridine

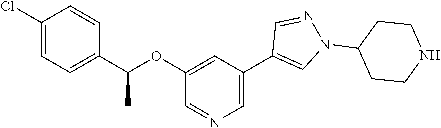 [SMILES: C[C@H](Oc1cncc(-c2cnn(C3CCNCC3)c2)c1)c1ccc(Cl)cc1]

Prepare in a manner substantially similar to (R)-3-(1-(4-chlorophenyl)ethoxy)-5-(1-(piperidin-4-yl)-1H-pyrazol-4-yl)pyridine. MS 383.30.

### Example 16

### Compound 25

### 3-((2-Methylphenyl)methoxy)-5-(1-(piperidin-4-yl)-1H-pyrazol-4-yl)pyridine

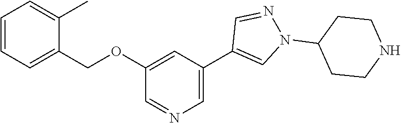 [SMILES: Cc1ccccc1COc1cncc(-c2cnn(C3CCNCC3)c2)c1]

### Intermediate 6 (Starting Material for Compound 25) (tert-butyl 4-(4-(5-((2-methylbenzyl)oxy)pyridin-3-yl)-1H-pyrazol-1-yl)piperidine-1-carboxylate)

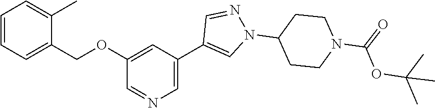 [SMILES: Cc1ccccc1COc1cncc(-c2cnn(C3CCN(C(=O)OC(C)(C)C)CC3)c2)c1]

To a stirred solution of tert-butyl 4-(4-(5-hydroxypyridin-3-yl)-1H-pyrazol-1-yl)piperidine-1-carboxylate (0.5 g, 1.45 mmol) in N,N-dimethylformamide (5 mL) was added dipotassium carbonate (602 mg, 3 eq., 4.36 mmol) then 1-(bromomethyl)-2-methylbenzene (403 mg, 1.5 eq., 2.18 mmol) was added. Reaction mixture was heated and stirred at 100° C. for 16 h. Reaction was monitored by TLC and LCMS. After completion, reaction mixture was quenched with water (15 ml) and extracted in ethyl acetate (50 mL). Organic layer was washed with ice cold water and brine solution to remove DMF then was dried over sodium sulphate and distilled to get crude. Crude was purified by combi-flash chromatography using 100-200 silica. Pure compound was eluted to 40% ethyl acetate in hexanes to afford light yellow gum which was further triturated with diethyl ether to afford tert-butyl 4-(4-(5-((2-methylbenzyl)oxy)pyridin-3-yl)-1H-pyrazol-1-yl)piperidine-1-carboxylate (230 mg, 492 μmol) as a white solid. MS 449.38.

### 3-((2-Methylphenyl)methoxy)-5-(1-(piperidin-4-yl)-1H-pyrazol-4-yl)pyridine (Compound 25)

To a stirred solution of tert-butyl 4-(4-(5-((2-methylbenzyl)oxy)pyridin-3-yl)-1H-pyrazol-1-yl)piperidine-1-carboxylate (100 mg, 223 μmol) was added trifluoracetic acid (0.5 mL) at 0° C. Then reaction mixture was stirred at room temperature for 3 h. Reaction was monitored by TLC and LCMS. After completion, reaction mixture was distilled up to dryness to get crude. Crude was purified by combi-flash chromatography using 100-200 silica. Pure compound was eluted to 15% MeOH in DCM to afford 3-((2-Methylphenyl)methoxy)-5-(1-(piperidin-4-yl)-1H-pyrazol-4-yl)pyridine as light green gum. MS 349.37.

### Example 17

### Compound 26

### 3-((2-chlorobenzyl)oxy)-5-(1-(piperidin-4-yl)-1H-pyrazol-3-yl)pyridine

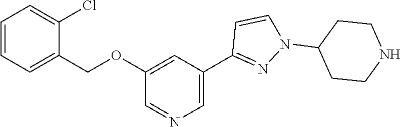 [SMILES: Clc1ccccc1COc1cncc(-c2ccn(C3CCNCC3)n2)c1]

Intermediate 7 (starting material for Intermediate 8, which is the starting material for Compound 26) (tert-butyl 4-(3-(5-hydroxypyridin-3-yl)-1H-pyrazol-1-yl)piperidine-1-carboxylate)

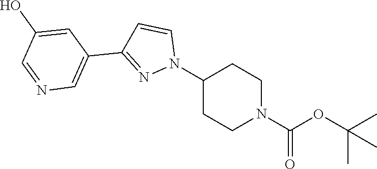 [SMILES: CC(C)(C)OC(=O)N1CCC(n2ccc(-c3cncc(O)c3)n2)CC1]

To a stirred solution of 5-bromopyridin-3-ol (1 g, 5.75 mmol) and tert-butyl 4-[3-(4,4,5,5-tetramethyl-1,3,2-dioxaborolan-2-yl)-1H-pyrazol-1-yl]piperidine-1-carboxylate (2.17 g, 5.75 mmol) in toluene (3 mL, 25.4 mmol), ethanol (3 mL, 51.4 mmol) and water (1 mL, 55.5 mmol), was added dipotassium carbonate (278 mg, 2 eq., 2.01 mmol) and reaction mixture was purged with nitrogen for 20 minutes. Then 1,1′-bis(diphenylphosphino)ferrocene-palladium(II)dichloride dichloromethane complex (469 mg, 0.1 eq., 575 μmol) was added and reaction mixture was heated at 100° C. and stirred for 16 h. Reaction mixture was monitored by TLC and LCMS. Reaction mixture was concentrated. Crude was purified by flash chromatography using 50% EtOAc in hexane as an eluent, desired fractions were concentrated to afford tert-butyl 4-[3-(5-hydroxypyridin-3-yl)-1H-pyrazol-1-yl]piperidine-1-carboxylate (0.3 g, 775 μmol, 13.5%) as an off-white solid. MS 345.25.

### Intermediate 8 (Starting Material for Compound 26) (tert-butyl 4-(3-(5-((2-chlorobenzyl)oxy)pyridin-3-yl)-1H-pyrazol-1-yl)piperidine-1-carboxylate)

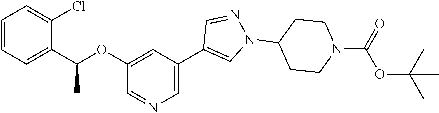 [SMILES: C[C@H](Oc1cncc(-c2cnn(C3CCN(C(=O)OC(C)(C)C)CC3)c2)c1)c1ccccc1Cl]

To a stirred solution of tert-butyl 4-(3-(5-hydroxypyridin-3-yl)-1H-pyrazol-1-yl)piperidine-1-carboxylate (250 mg, 726 μmol) and 1-(bromomethyl)-2-chlorobenzene (149 mg, 726 μmol) in dimethylformamide (5 mL, 64.6 mmol), was added dipotassium carbonate (301 mg, 3 eq., 2.18 mmol) portion wise and reaction mixture heated at 100° C. for 16 h. After completion (TLC and LCMS monitoring), the reaction mixture was diluted with water, extracted with EtOAc, washed with brine, dried over anhydrous sodium sulfate, filtered and concentrated. Crude was purified by flash chromatography using 40% EtOAc in hexane as an eluent, desired fractions were concentrated to afford tert-butyl 4-(3-(5-((2-chlorobenzyl)oxy)pyridin-3-yl)-1H-pyrazol-1-yl)piperidine-1-carboxylate (0.1 g, 198 μmol) as an off white solid. MS 469.25.

### 3-((2-chlorobenzyl)oxy)-5-(1-(piperidin-4-yl)-1H-pyrazol-3-yl)pyridine (Compound 26)

To a stirred solution of tert-butyl 4-(3-(5-((2-chlorobenzyl)oxy)pyridin-3-yl)-1H-pyrazol-1-yl)piperidine-1-carboxylate (0.2 g, 426 μmol) in dichloromethane (5 mL, 78.1 mmol), was added trifluoroacetic acid (0.4 mL, 4 mmol) drop wise at 0° C. and reaction mixture was stirred at room temperature for 3 hours. After completion (TLC and LCMS monitoring), reaction mixture was diluted with water and extracted with dichloromethane. Organic layer was washed with brine and dried over sodium sulphate and concentrated. Crude was purified by prep HPLC using TFA as a buffer to afford the TFA salt of 3-((2-chlorobenzyl)oxy)-5-(1-(piperidin-4-yl)-1H-pyrazol-3-yl)pyridine (20 mg, 40.6 μmol) as an off white solid. MS 369.25.

### Example 18

### Compound 27

### 3-((2-chlorobenzyl)oxy)-5-(3-(piperidin-4-yl)-1H-pyrazol-1-yl)pyridine

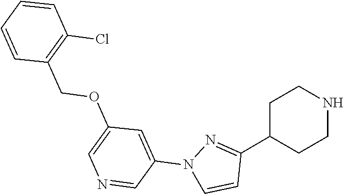 [SMILES: Clc1ccccc1COc1cncc(-n2ccc(C3CCNCC3)n2)c1]

### Intermediate 9 (starting material for Intermediate 10, which is the starting material for Compound 27) (3-bromo-5-((2-chlorobenzyl)oxy)pyridine)

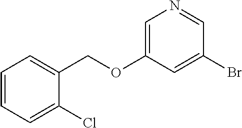 [SMILES: Clc1ccccc1COc1cncc(Br)c1]

To a stirred solution of 5-bromopyridin-3-ol (5.28 g, 30.3 mmol) in N,N-dimethylformamide (100 mL, 1.29 mol) was added cesium carbonate (10.9 g, 33.4 mmol) and 2-chlorobenzyl bromide (3.94 mL, 30.3 mmol). The reaction mixture was stirred at room temperature for 1 hour. After the reaction was complete (monitored by LCMS), the mixture was diluted with water (100 mL) and the resulting mixture was extracted with methyl tert-butyl ether (3×75 mL). The combined extracts were washed with water (100 mL) and brine (100 mL), dried over anhydrous sodium sulfate, filtered and concentrated. The residue was purified by flash chromatography using a gradient of 0% to 15% ethyl acetate in hexane as an eluent. Desired fractions were concentrated to afford 3-bromo-5-((2-chlorobenzyl)oxy)pyridine (5.3 g, 17.8 mmol) as a white solid. MS 298.15.

### Intermediate 10 (Starting Material for Compound 27) (tert-butyl 4-(1-(5-((2-chlorobenzyl)oxy)pyridin-3-yl)-1H-pyrazol-3-yl)piperidine-1-carboxylate)

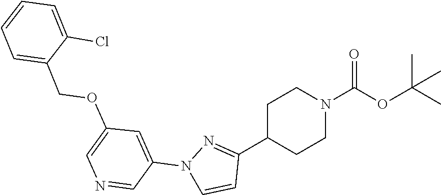 [SMILES: CC(C)(C)OC(=O)N1CCC(c2ccn(-c3cncc(OCc4ccccc4Cl)c3)n2)CC1]

To a reaction vessel was added tert-butyl 4-(1H-pyrazol-3-yl)piperidine-1-carboxylate (210 mg, 836 μmol), 3-bromo-5-((2-chlorobenzyl)oxy)pyridine (498 mg, 1.67 mmol), copper(I) iodide (27 mg, 142 μmol), potassium carbonate (294 mg, 2.13 mmol), potassium phosphate tribasic (289 mg, 1.36 mmol), acetonitrile (9 mL, 172 mmol), and (1R,2R)—N1,N2-dimethylcyclohexane-1,2-diamine (132 μmol, 836 μmol). The vessel was purged with nitrogen, sealed and heated 120° C. for 4 hours. After the reaction was complete (monitored by LCMS), the mixture was cooled to room temperature and diluted with saturated aqueous ammonium chloride (5 mL). This mixture was extracted with ethyl acetate (3×5 mL). The combined extracts were washed with saturated aqueous ammonium chloride (2 mL), dried over anhydrous sodium sulfate, filtered and concentrated. The residue was purified by flash chromatography using a gradient of 0% to 50% ethyl acetate in hexane as an eluent. Desired fractions were concentrated to afford tert-butyl 4-(1-(5-((2-chlorobenzyl)oxy)pyridin-3-yl)-1H-pyrazol-3-yl)piperidine-1-carboxylate (341 mg, 727 μmol) as clear colorless oil. MS 469.46.

### 3-((2-chlorobenzyl)oxy)-5-(3-(piperidin-4-yl)-1H-pyrazol-1-yl)pyridine (Compound 27)

To a stirred solution of tert-butyl 4-(1-(5-((2-chlorobenzyl)oxy)pyridin-3-yl)-1H-pyrazol-3-yl)piperidine-1-carboxylate (280 mg, 597 μmol) in dichloromethane (3 mL, 47.1 mmol) was added 4 N hydrochloric acid in dioxane (3 mL, 12 mmol) at room temperature. After 1 hour the reaction was complete (monitored by LCMS) and white solids settled to the bottom of the vessel. The supernatant was removed and the remaining solids were washed with dichloromethane (3×5 mL). The remaining solids were dried in vacuo to afford 3-((2-chlorobenzyl)oxy)-5-(3-(piperidin-4-yl)-1H-pyrazol-1-yl)pyridine (259 mg, 586 μmol) as a white solid. MS 369.38.

### Example 19

### Compound 28

### 3-((2-chlorobenzyl)oxy)-5-(4-(piperidin-4-yl)-1H-pyrazol-1-yl)pyridine

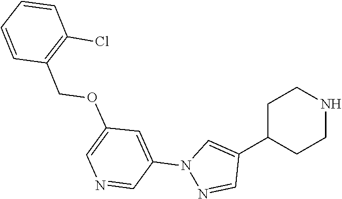 [SMILES: Clc1ccccc1COc1cncc(-n2cc(C3CCNCC3)cn2)c1]

### Intermediate 11 (Starting Material for Compound 28) (tert-butyl 4-(1-(5-((2-chlorobenzyl)oxy)pyridin-3-yl)-1H-pyrazol-4-yl)piperidine-1-carboxylate)

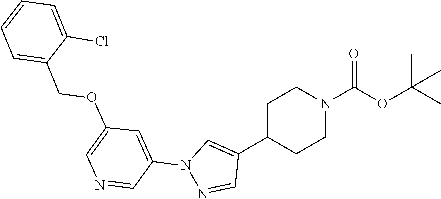 [SMILES: CC(C)(C)OC(=O)N1CCC(c2cnn(-c3cncc(OCc4ccccc4Cl)c3)c2)CC1]

To a reaction vessel was added tert-butyl 4-(1H-pyrazol-4-yl)piperidine-1-carboxylate (100 mg, 398 μmol), 3-bromo-5-((2-chlorobenzyl)oxy)pyridine (238 mg, 796 μmol), copper(I) iodide (12 mg, 63.0 μmol), potassium carbonate (137 mg, 995 μmol), potassium phosphate tribasic (135 mg, 637 μmol), acetonitrile (4 mL, 76.5 mmol), and (1R,2R)—N1,N2-dimethylcyclohexane-1,2-diamine (63 μmol, 400 μmol). The vessel was purged with nitrogen, sealed and heated 120° C. for 5 hours. After the reaction was complete (monitored by LCMS), the mixture was cooled to room temperature and diluted with saturated aqueous ammonium chloride (3 mL). This mixture was extracted with ethyl acetate (3×3 mL). The combined extracts were washed with water (3×1 mL), dried over anhydrous sodium sulfate, filtered and concentrated. The residue was purified by flash chromatography using a gradient of 0% to 40% ethyl acetate in hexane as an eluent. Desired fractions were concentrated to afford tert-butyl 4-(1-(5-((2-chlorobenzyl)oxy)pyridin-3-yl)-1H-pyrazol-4-yl)piperidine-1-carboxylate (169 mg, 360 μmol) as a yellow oil. MS 469.50.

### 3-((2-chlorobenzyl)oxy)-5-(4-(piperidin-4-yl)-1H-pyrazol-1-yl)pyridine (Compound 28)

To a stirred solution of tert-butyl 4-(1-(5-((2-chlorobenzyl)oxy)pyridin-3-yl)-1H-pyrazol-4-yl)piperidine-1-carboxylate (150 mg, 320 μmol) in dichloromethane (2 mL, 31.4 mmol) was added 4 N hydrochloric acid in dioxane (0.5 mL, 2 mmol) at room temperature. After 2 hours additional 4 N hydrochloric acid in dioxane (0.5 mL, 2 mmol) was added and the reaction mixture was stirred overnight. After completion (monitored by LCMS) the solution was concentrated in vacuo and washed with methyl tert-butyl ether (2×2 mL). The remaining solids were dried in vacuo to afford the HCl salt of 3-((2-chlorobenzyl)oxy)-5-(4-(piperidin-4-yl)-1H-pyrazol-1-yl)pyridine (118 mg, 267 μmol) as a white solid. MS 369.38.

### Example 20

Compound 29

### 4-(4-(3-((2-chlorobenzyl)oxy)phenyl)-1H-pyrazol-1-yl)piperidine

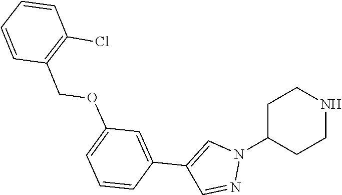 [SMILES: Clc1ccccc1COc1cccc(-c2cnn(C3CCNCC3)c2)c1]

### Intermediate 12 (Starting Material for Intermediate 13, which is the Starting Material for Compound 29) (tert-butyl 4-(4-(3-hydroxyphenyl)-1H-pyrazol-1-yl)piperidine-1-carboxylate)

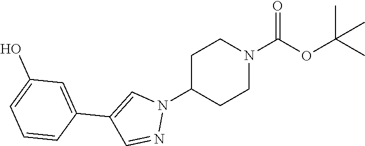 [SMILES: CC(C)(C)OC(=O)N1CCC(n2cc(-c3cccc(O)c3)cn2)CC1]

To a stirred solution of 3-bromophenol (1 g, 5.78 mmol) in toluene (80.5 mL, 68 mmol) and ethanol (8.05 mL, 138 mmol) were added dipotassium carbonate (1.6 g, 2 eq., 11.6 mmol) and tert-butyl 4-[4-(4,4,5,5-tetramethyl-1,3,2-dioxaborolan-2-yl)-1H-pyrazol-1-yl]piperidine-1-carboxylate (2.62 g, 1.2 eq., 6.94 mmol). Then reaction mixture was purged with N₂ for 10 minutes, then added tetrakis(triphenylphosphine)palladium(0) (334 mg, 0.05 eq., 289 μmol) the resulting reaction mass heated at 100° C. for 16 hours. After completion (TLC and LCMS monitoring, diluted with water, extracted with ethyl acetate, washed with water, brine, dried over anhydrous Na₂SO₄, filtered and concentrated. Crude was purified by flash chromatography using 0-40% ethyl acetate in n-heptane as an eluent, desired fractions were concentrated to afford tert-butyl 4-(4-(3-hydroxyphenyl)-1H-pyrazol-1-yl)piperidine-1-carboxylate (700 mg, 2.04 mmol) as a white solid. MS 344.36.

### Intermediate 13 (Starting Material for Compound 29) (tert-butyl 4-(4-(3-((2-chlorobenzyl)oxy)phenyl)-1H-pyrazol-1-yl)piperidine-1-carboxylate)

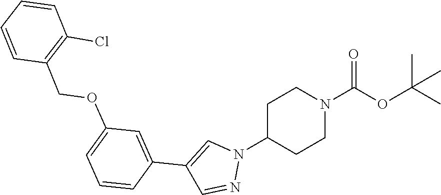 [SMILES: CC(C)(C)OC(=O)N1CCC(n2cc(-c3cccc(OCc4ccccc4Cl)c3)cn2)CC1]

To a stirred solution of tert-butyl 4-(4-(3-hydroxyphenyl)-1H-pyrazol-1-yl)piperidine-1-carboxylate (500 mg, 1.4 eq., 1.45 mmol) in dimethylformamide (5 mL, 64.6 mmol) was added dipotassium carbonate (420 mg, 3 eq., 3.04 mmol) and 1-(bromomethyl)-2-chlorobenzene (312 mg, 1.5 eq., 1.52 mmol) then reaction was heated at 100° C. for 16 hours. After completion (TLC and LCMS monitoring), mixture was diluted with cold water, extracted with ethyl acetate, washed with water, brine, dried over Na₂SO₄, filtered and concentrated. Crude was purified by flash chromatography using 0-20% ethyl acetate as an eluent, desired fractions were concentrated to afford tert-butyl 4-(4-(3-((2-chlorobenzyl)oxy)phenyl)-1H-pyrazol-1-yl)piperidine-1-carboxylate (250 mg, 534 μmol) as an off white solid. MS 468.35.

### 4-(4-(3-((2-chlorobenzyl)oxy)phenyl)-1H-pyrazol-1-yl)piperidine (Compound 29)

To a stirred solution of tert-butyl 4-(4-(3-((2-chlorobenzyl)oxy)phenyl)-1H-pyrazol-1-yl)piperidine-1-carboxylate (130 mg, 277 μmol) in dichloromethane (4.85 mL, 75.8 mmol) were added trifluoroacetic acid (2 mL) dropwise at 0° C. then reaction stirred at RT for 3 h. After the reaction was complete (monitored by LCMS), the mixture was concentrated and resulting solid was triturated with diethyl ether to afford the TFA salt of 4-(4-(3-((2-chlorobenzyl)oxy)phenyl)-1H-pyrazol-1-yl)piperidine trifluoroacetate (102 mg, 245 μmol, 88%) as a white solid. MS 368.30.

### Example 21

### Malachite Green Enzyme Assay

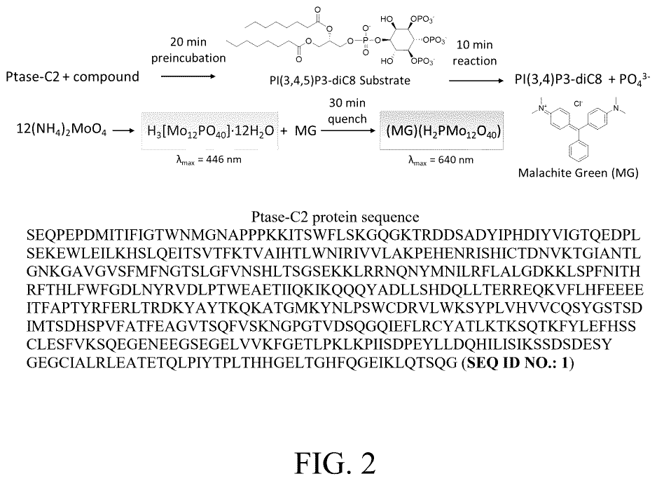

Enzymatic inhibitory potencies (IC₅₀) (see table below) were determined using PI(3,4,5)P3-diC8 as a substrate at 25° C. in 50 mM HEPES buffer (pH7.4, 150 mM NaCl, 2 mM MgCl2). See FIG. 2. Compounds diluted in DMSO were added to 384-well plates. Enzyme solution was added. After a 20 min incubation period, the reaction was initiated by addition of PI(3,4,5)P3-diC8. Final compound concentrations ranged from 50 nM to 950 μM. Final reaction concentrations for the PI(3,4,5)P3-diC8 substrate and the enzyme were 52 μM and 10 nM respectively. The reaction was quenched after 10 min by adding Malachite BioMol Green (Enzo Lifesciences, PA, USA). Plates were then incubated for 30 min at room temperature. Absorbance (620 nm) was measured using a SpectraMax Me5 Microplate Reader (Molecular Devices, LLC, USA). IC₅₀ values were calculated by fitting absorbance versus inhibitor concentration and are reported as the geometric mean and standard error of mean with the number of repeats (n) indicated. Compounds and activities are as depicted in tables 1 and 2 above.

### Example 22

### Cellular Thermal Shift Assay (CETSA)

A split Nano Luciferase assay (SplitLuc CETSA) was used to demonstrate target engagement of SHIP1 inhibitors in a physiologically relevant cellular context by quantifying changes in the thermal stability of a HiBit-labeled full length SHIP1 protein in intact cells. See Martinez et al. (2018) Sci Rep 8: p. 9472; Oh-Hashi et al. (2017) Biochem Biophys Rep 12: pp. 40-45. This assay was run in the following two formats with HMC3/HiBit-INPP5D stably transfected cells. Results are depicted in the table below.

Thermal shift: cells were treated with 40 μM compound for 60 min, then heated with a temperature gradient covering 38-52° C. for 3 min before luminescence detection. Mean and standard deviation of control Tm were determined to generate a “Mean+3SD” ΔTm, which was used as threshold to determine a significant ΔTm of compound from control. When the difference of ΔTm of compound treated cells from control Tm>3SD, the compound was considered positive for target engagement, otherwise negative. SD=average standard deviation.

Compound dose response: run at target Tm (44.2° C. for SHIP1) with compound dosing from 80 uM or 100 uM with 1:3 serial dilutions to generate an 8-point curve. Cells were treated for 60 min before being heated at target Tm for 3 min before luminescence detection. The concentration that induced a half-maximum response (AC₅₀) was calculated using a four-parameter logistic curve regression model with change at highest concentration noted when difference from control >3SD. Activities are shown in table 3 below.

### Example 23

### Phosphoakt Assay.

THP1 cells were treated with inhibitors for 90 minutes and then the levels of phosphorylated and total AKT (pAKT/tAKT) were detected using the Perkin Elmer Alpha SureFire Ultra Multiplex PhosphoAKT (S473) kit according to the manufacturer's directions. IC₅₀ values were calculated by fitting the ratio of pATK/tAKT versus inhibitor concentration.

### Example 24

pHrodo-Myelin Phagocytosis/Cell Health Assay with Microglial Cells

This 384-well plate high content imaging assay was developed to quantify phagocytosis and cell health simultaneously using either BV2 or HMC3 immortalized microglial cell lines or primary microglia isolated from mouse brain. Cells were cultured in DMEM GlutaMax media (ThermoFisher) containing 10% FBS and Pen-Strep in 37° C. 5% CO₂ incubator. Day 1: Cells were plated (Corning Falcon 384 well Optilux Black and clear bottom plates for imaging) with BV2 at 400 cells/45 μl/well, HMC3 at 600 cells/45 μl/well, or primary at 2000 cell/45 μl/well. Day 2: Cells were treated with 10× serially diluted compounds in a dose range of 60 μM to 3 nM for 48 hrs at 37° C. Day 3: Cells were seeded with pHrodo-myelin (for total 20 hrs) 24 hrs after starting compound treatment. The pHrodo-myelin stocks were at 1 mg/ml (protein equivalent) stored in −20° C. or −80° C. freezer. Stocks were thawed and diluted with culture media into 10× seeding solution (50 μg/ml) and added 5 μl/well to 384 well cell plate. Day 4: Cell staining and imaging. Nuclear staining solution was prepared by adding 1 μl of 10 mg/mL Hoechst-33342 to every 1 ml culture media that will be added to cell plate at 20 μl/well. The final concentration of Hoechst-33342 to cells was about 2.5 μg/ml. Cell plates were incubated for >30 min at 37° C. before imaging. Cell plates were scanned with an ArrayScan automatic high content imaging system using a 10× objective lens, 4 fields/well collected. Three measurements were obtained 1) mean total phagocytosis spot intensity per cell, 2) total cell counts per well, and 3) mean average nuclear intensity per cell for cell health. Apoptotic cells showed nuclear intensity increase (early apoptosis) or decrease (later apoptosis). Activities are shown in table 3 below.

### Example 25

### Activity of Compound 9 in Primary Mouse Microglia

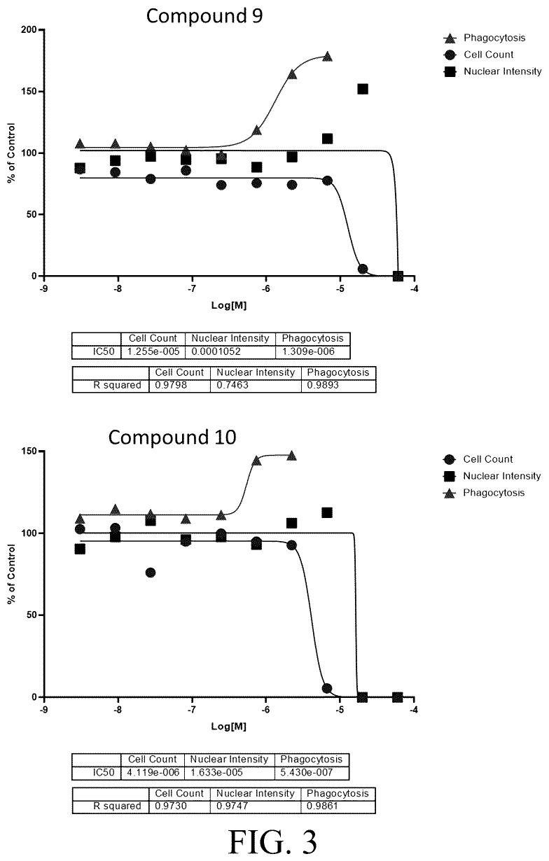

Cortical tissue from C57BL/6J neonatal mice (P0-P3) was homogenized in Dulbecco's Modified Eagle Medium (DMEM), filtered through 250 and 100 μm mesh, and cultured in Advanced DMEM/F12 supplemented with 10% fetal bovine serum, 1× GlutaMAX and 1× Penicillin/Streptomycin. At 21 days in vitro (DIV), the cultures were subjected to mild trypsinization using 0.083% Trypsin-EDTA in DMEM for 30 mins to detach an intact layer of astrocytes. The microglia attached to the bottom were used as described in Example 13 to measure myelin phagocytosis and cell health. The results are depicted in FIG. 3.

### Example 26

### Activity of Compound 10 in Primary Mouse Microglia

Primary Mouse Microglia of compound 10 was obtained in a similar manner to compound 9. The results are depicted in FIG. 3.

### Example 27

### Pharmacokinetics of Compound 9 in C57BL/6J Mice

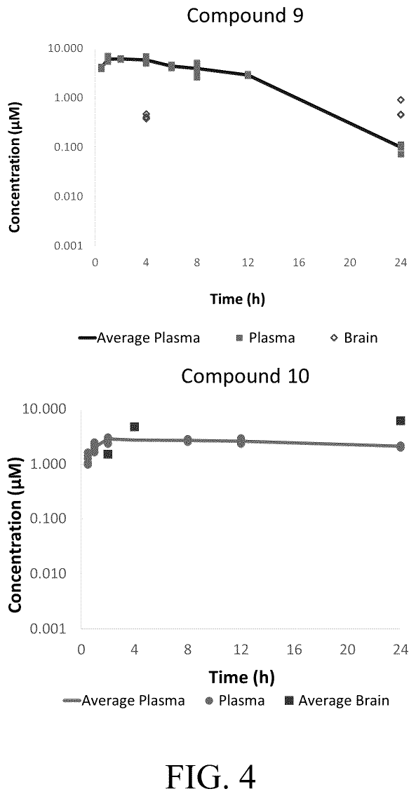

Compound was formulated in hydroxyethylcellulose (1%)/Tween 80 (0.25%)/antifoam (0.050%)/water (purified) at a concentration of 5 mg/ml. 100 mg/kg, 20 ml/kg was dosed via oral gavage. Plasma exposures at 0.25, 0.5, 1, 2, 8 hr and terminal at 24 hrs were obtained. Brain exposures were obtained at terminal 4 and 24 hrs. The results are depicted in FIG. 4.

### Example 28

### Pharmacokinetics of Compound 10 in C57BL/6J Mice

Pharmacokinetics of compound 10 in C57BL/6J Mice were obtained in a similar manner as compound 9. The results are depicted in FIG. 4.

TABLE 3

| | | | CETSA | | | |
|---|---|---|---|---|---|---|
| | | CETSA | Min | BV2 | HMC3 | |
| | CETSA | AC50 | Inhibition | Phagocytosis | Phagocytosis | PhosphoAKT |
| Compound | SP | (uM) | (%) | EC₅₀ | EC₅₀ | IC₅₀ |
| 1 | N | NC | 74.7 | 2.33 (4) | 15.9 (4) | NC* |
| 2 | N | NC | 70.6 | 1.56 (4) | 1.44 (3) | NT* |
| 3 | N | 77.0 | 60.8 | 1.03 (1) | 4.19 (1) | 31.2 |
| 4 | N | >80 | 69.2 | 3.23 (2) | 2.01 (2) | 31.6 (2) |
| 5 | N | NT | NT | NC (1) | 3.72 (1) | NT |
| 7 | Y* | NC | 66.3 | 0.756 (1) | 2.43 (1) | NT |
| 9 | Y | 55.0 | 16.0 | 0.741 (4) | 0.584 (6) | NT |
| 10 | Y | 53.8 | 10.2 | 1.75 (5) | 0.308 (5) | 6.52 (3) |
| 15 | N | NC | 66.7 | 2.26 (2) | 2.10 (2) | 17.9 (1) |
| 16 | N | 79.6 | 64.5 | 1.17 (2) | 2.07 (2) | >60 (1) |
| 19 | N | NC | 72.1 | 0.958 (1) | NC (1) | NC* |
| 21 | Y | 54.7 | 24.9 | 0.395 (1) | 0.722 (2) | NT |
| 22 | Y | 43.4 | 24.6 | NC (1) | NC (1) | NT |
| 23 | Y | 50.6 | 8.15 | 2.60 (2) | 0.145 (2) | 4.06 (2) |
| 25 | Y | 83.6 | 24.9 | NC (2) | 1.35 (2) | 58.4 (1) |
| 26 | Y | 87.4 | 14.6 | NC (2) | NC (2) | 7.72 (2) |
| 27 | Y | 57.1 | 10.1 | NC (3) | NC (3) | >60 (1) |
| 28 | Y | 55.5 | 5.98 | NC (2) | NC (2) | 5.57 (2) |
| 29 | N | 55.0 | 28.4 | NC (3) | NC (3) | 6.55 (2) |

Y = ΔTm difference of 40 μM compound treated cells from control is Tm > 3SD;
NT = Not Tested;
NC = Not Calculated

### SUMMARY

The SHIP1 phosphatase is generally understood as a brake on microglia activation downstream from TREM2 and Fc receptors. SHIP1 inhibitors described herein increase phagocytosis of mouse (BV2) and human (HMC3) microglia cell lines between 1.2- to 2-fold over baseline at μM concentrations with minimal effects on cell health. In primary mouse microglia Compound 9 increased phagocytosis 1.8-fold with an EC₅₀ of 1.3 μM. Compound 10 increased phagocytosis 1.5-fold with an EC₅₀ of 0.54 μM. Exposures of compound 9 in C57BL/6J− mice dosed orally at 100 mg/kg achieved exposures of 5 μM in plasma and 0.5 μM in brain indicating sufficient target engagement in vivo may be obtained for pharmacodynamic studies. Exposures of compound 10 achieved exposures of 2 μM in plasma and 6 μM in brain.

As will be appreciated from the descriptions herein, a wide variety of aspects and embodiments are contemplated by the present disclosure, examples of which include, without limitation, the aspects and embodiments listed below:

The current disclosure provides methods and compounds directed to inhibiting SHIP1 in order to activate microglia. The methods and compounds disclosed herein allow for the inhibition of SHIP1 early in neurodegenerative diseases leading to increased microglial protective functions and reduce the rate of disease progression and cognitive decline, for example, in Alzheimer's patients.

More specifically, the present disclosure provides:

A compound of formula I:

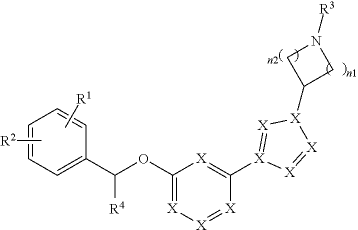

wherein each X is independently C or N;

$$n 1 + n 2 = 4 ;$$

R1, R2 are independently H, F, Cl, Br or methyl;
R3 is H, amide, carbamate, or alkyl; and
R4 is H or CH₃,
or pharmaceutically acceptable salt thereof.

Alone or in combination with any other embodiment, a method for treating a neurodegenerative disorder or related condition in a patient, comprising administering to a patient in need thereof an effective amount of a compound of formula I.

Alone or in combination with any other embodiment, a method of treating the progression of a neurodegenerative disorder or related condition in a patient, comprising administering to a patient in need of such treatment an effective amount of a compound of formula I.

Alone or in combination with any other embodiment, a method of preventing a neurodegenerative disorder or related condition in a patient, comprising administering to a patient in need of such treatment an effective amount of a compound of formula I.

Alone or in combination with any other embodiment, the compound or its pharmaceutically acceptable salt thereof, is formulated in a pharmaceutical composition further comprising one or more pharmaceutically acceptable carriers, diluents or excipients.

Alone or in combination with any other embodiment, the method further comprises the administration of one or more additional therapeutic agents.

Alone or in combination with any other embodiment, a pharmaceutical composition comprising a compound of formula I further comprises one or more pharmaceutically acceptable carriers, diluents or excipients.

Alone or in combination with any other embodiment, the pharmaceutical composition is administered to a patient in need thereof for the treatment or prevention of a neurological disorder or related condition or to inhibit the progression of said neurological disorder or related condition.

Alone or in combination with any other embodiment, the pharmaceutical composition further comprises one or more one or more additional therapeutic agents.

Alone or in combination with any other embodiment, a compound of formula II:

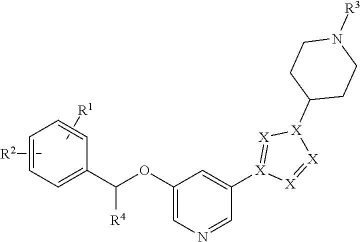

wherein each X is independently C or N;
R1, R2 are independently H, F, Cl, Br or methyl;
R3 is H, amide, carbamate, or alkyl; and
R4 is H or CH₃,
or its pharmaceutically acceptable salt thereof, is used for the manufacture of a medicament for the treatment or prevention of a neurodegenerative disorder or related condition or to inhibit the progression of said neurological disorder or related condition.

Alone or in combination with any other embodiment, a compound of formula III:

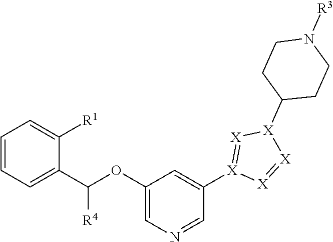

wherein each X is independently C or N;
R1, R2 are independently H, F, Cl, Br or methyl;
R3 is H, amide, carbamate, or alkyl; and
R4 is H or CH₃,
or its pharmaceutically acceptable salt thereof, is used for the manufacture of a medicament for the treatment or prevention of a neurodegenerative disorder or related condition or to inhibit the progression of said neurological disorder or related condition.

Alone or in combination with any other embodiment, a compound of formula IV:

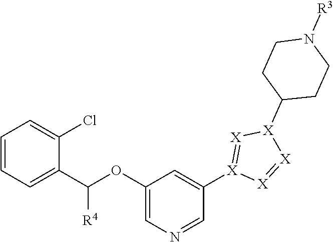

wherein each X is independently C or N;
R1, R2 are independently H, F, Cl, Br or methyl;
R3 is H, amide, carbamate, or alkyl; and
R4 is H or CH₃,
or its pharmaceutically acceptable salt thereof, is used for the manufacture of a medicament for the treatment or prevention of a neurodegenerative disorder or related condition or to inhibit the progression of said neurological disorder or related condition.

Alone or in combination with any other embodiment, a compound selected from

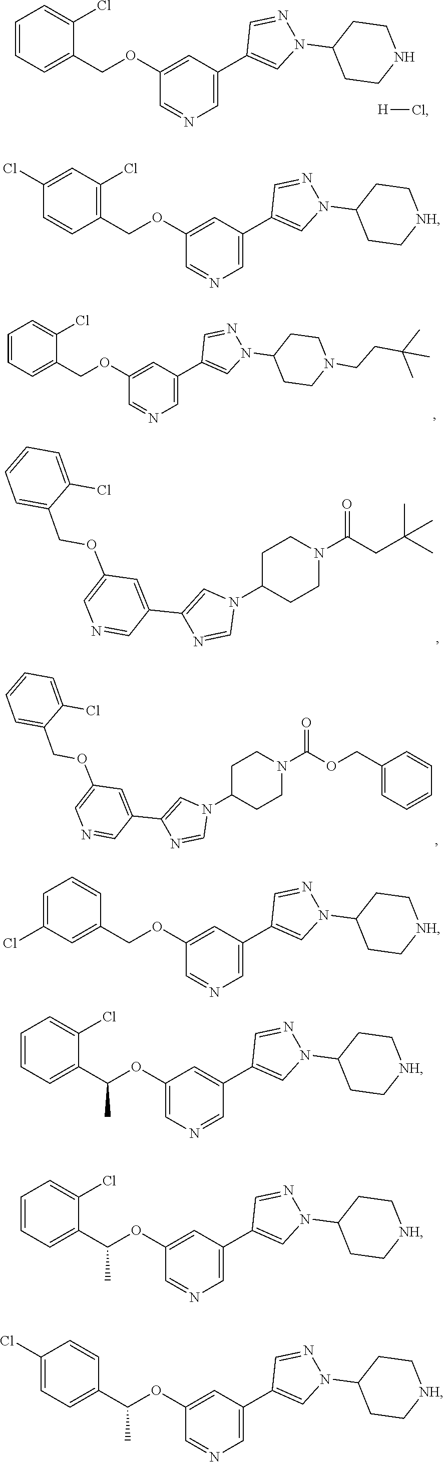

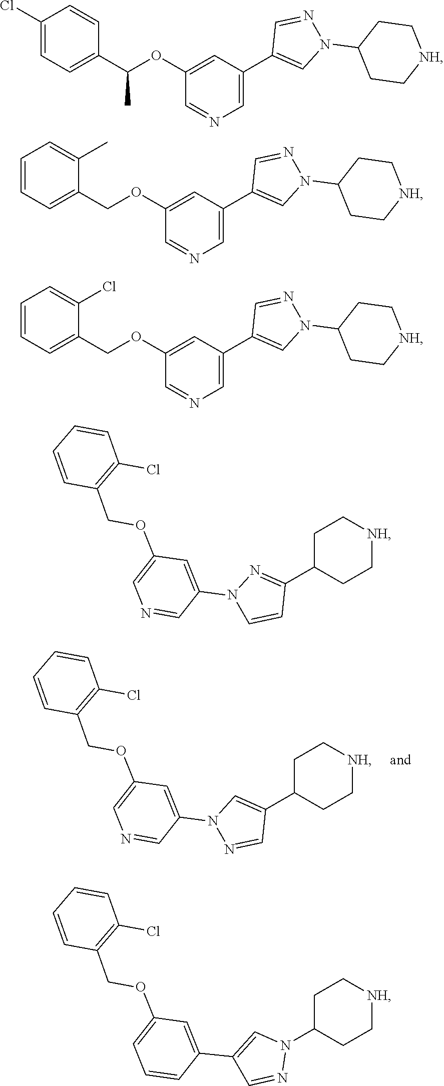

or its pharmaceutically acceptable salt thereof, is used for the manufacture of a medicament for the treatment or prevention of a neurodegenerative disorder or related condition or to inhibit the progression of said neurological disorder or related condition.

Alone or in combination with any other embodiment, a compound

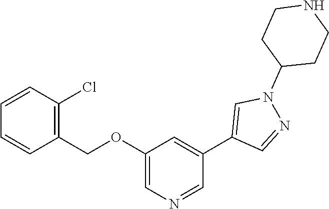 [SMILES: Clc1ccccc1COc1cncc(-c2cnn(C3CCNCC3)c2)c1]

or its pharmaceutically acceptable salt thereof, is used for the manufacture of a medicament for the treatment or prevention of a neurodegenerative disorder or related condition or to inhibit the progression of said neurological disorder or related condition.

Alone or in combination with any other embodiment, the neurodegenerative disorder or related condition is selected from the group consisting of Alzheimer's disease, Alzheimer's disease-related dementias or mild cognitive impairment.

Alone or in combination with any other embodiment, the Alzheimer's disease-related dementia is selected from the group consisting of Lewy body dementia (LBD), frontotemporal degeneration (FTD), vascular cognitive impairment and dementia (VCID), and multiple etiology dementias.

Alone or in combination with any other embodiment, is a method of synthesizing a compound of formula I.

Alone or in combination with any other embodiment, the compound of formula I is:

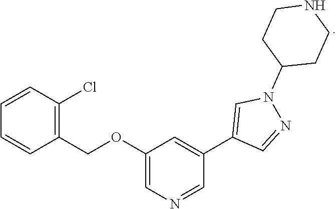 [SMILES: Clc1ccccc1COc1cncc(-c2cnn(C3CCNCC3)c2)c1]

Alone or in combination with any other embodiment, methods for treating Alzheimer's disease in a patient, comprise administering to a patient in need thereof a compound of the present disclosure or a pharmaceutically acceptable salt thereof.

Alone or in combination with any other embodiment, methods for treating Alzheimer's disease-related dementias in a patient, comprise administering to a patient in need thereof a compound of the present disclosure or a pharmaceutically acceptable salt thereof.

Alone or in combination with any other embodiment, methods for treating the progression of mild cognitive impairment to Alzheimer's disease or related dementias in a patient, comprise administering to a patient in need of such treatment an effective amount of a compound of the present disclosure.

Alone or in combination with any other embodiment, methods for preventing Alzheimer's disease or Alzheimer's disease-related dementias, comprise administering to a patient in need of such treatment an effective amount of a compound of the present disclosure.

Alone or in combination with any other embodiment, pharmaceutical compositions comprise a compound of the disclosure, or a pharmaceutically acceptable salt thereof, in combination with one or more pharmaceutically acceptable carriers, diluents or excipients.

Alone or in combination with any other embodiment, pharmaceutical compositions comprise a compound of the disclosure, or a pharmaceutically acceptable salt thereof, in combination with one or more additional therapeutic agents.

Alone or in combination with any other embodiment, pharmaceutical compositions comprise a compound of the disclosure, or a pharmaceutically acceptable salt thereof, for the treatment of Alzheimer's disease, in combination with one or more pharmaceutically acceptable carriers, diluents or excipients.

Alone or in combination with any other embodiment, pharmaceutical compositions for the treatment of Alzheimer's disease-related dementias, comprise a compound of the disclosure, or a pharmaceutically acceptable salt thereof, in combination with one or more carriers, diluents, or pharmaceutically acceptable excipients.

Alone or in combination with any other embodiment, pharmaceutical compositions for the prevention of Alzheimer's disease and Alzheimer's disease-related dementias, comprise a compound of the disclosure, or a pharmaceutically acceptable salt thereof, in combination with one or more carriers, diluents, or pharmaceutically acceptable excipients.

Alone or in combination with any other embodiment, a compound of the disclosure, or a pharmaceutically acceptable salt thereof, is used in therapy, in particular for the treatment of Alzheimer's disease.

Alone or in combination with any other embodiment, a compound of the disclosure, or a pharmaceutically acceptable salt thereof, is used in the treatment of Alzheimer's disease.

Alone or in combination with any other embodiment, a compound of the disclosure, or a pharmaceutically acceptable salt thereof, is used for the manufacture of a medicament for the treatment of Alzheimer's disease.

Alone or in combination with any other embodiment, a compound of the disclosure, or a pharmaceutically acceptable salt thereof, is used in therapy, in particular for the treatment of Alzheimer's disease-related dementias.

Alone or in combination with any other embodiment, a compound of the disclosure, or a pharmaceutically acceptable salt thereof, is used in the treatment of Alzheimer's disease-related dementias.

Alone or in combination with any other embodiment, a compound of the disclosure, or a pharmaceutically acceptable salt thereof, is used for the manufacture of a medicament for the treatment of Alzheimer's disease-related dementias.

Alone or in combination with any other embodiment, a compound of the disclosure, or a pharmaceutically acceptable salt thereof, is used for the prevention of Alzheimer's disease and Alzheimer's disease-related dementias.

Alone or in combination with any other embodiment, a compound of the disclosure, or a pharmaceutically acceptable salt thereof, is used for the prevention of Alzheimer's disease and Alzheimer's disease-related dementias.

Alone or in combination with any other embodiment, a compound of the disclosure, or a pharmaceutically acceptable salt thereof, is used for the manufacture of a medicament for use in the prevention of Alzheimer's disease and Alzheimer's disease-related dementias.

The present disclosure also encompasses intermediates and processes useful for the synthesis of a compound of the present disclosure.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A compound of formula I: 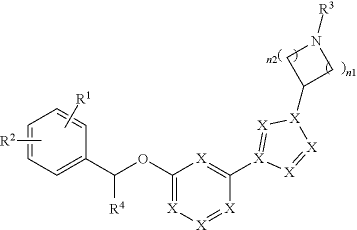
wherein each X is independently C or N;
n1+n2=4;
R1, R2 are independently H, F, Cl, Br or methyl;
R3 is H, amide, carbamate, or alkyl; and
R4 is H or CH₃,
or pharmaceutically acceptable salt thereof.

2. A method for treating a neurodegenerative disorder or related condition in a patient, comprising administering to a patient in need thereof an effective amount of a compound of claim 1
or pharmaceutically acceptable salt thereof.

3. A method of treating the progression of a neurodegenerative disorder or related condition in a patient, comprising administering to a patient in need of such treatment an effective amount of a compound of claim 1
or pharmaceutically acceptable salt thereof.

4. A method of preventing a neurodegenerative disorder or related condition in a patient, comprising administering to a patient in need of such treatment an effective amount of a compound of claim 1
or pharmaceutically acceptable salt thereof.

5. The method of claim 2, wherein the compound or its pharmaceutically acceptable salt thereof, is formulated in a pharmaceutical composition further comprising one or more pharmaceutically acceptable carriers, diluents or excipients.

6. The method of claim 3, wherein the compound or its pharmaceutically acceptable salt thereof, is formulated in a pharmaceutical composition further comprising one or more pharmaceutically acceptable carriers, diluents or excipients.

7. The method of claim 4, wherein the compound or its pharmaceutically acceptable salt thereof, is formulated in a pharmaceutical composition further comprising one or more pharmaceutically acceptable carriers, diluents or excipients.

8. The method of claim 5, further comprising the administration of one or more additional therapeutic agents.

9. The method of claim 6, further comprising the administration of one or more additional therapeutic agents.

10. The method of claim 7, further comprising the administration of one or more additional therapeutic agents.

11. A pharmaceutical composition comprising a compound of claim 1
or its pharmaceutically acceptable salt thereof, further comprising in combination with one or more pharmaceutically acceptable carriers, diluents or excipients.

12. The pharmaceutical composition of claim 11, administered to a patient in need thereof for the treatment or prevention of a neurological disorder or related condition or to inhibit the progression of said neurological disorder or related condition.

13. The pharmaceutical composition of claim 11, further comprising one or more one or more additional therapeutic agents.

14. A method of treatment or prevention of a neurodegenerative disorder or related condition or to inhibit the progression of said neurological disorder or related condition, comprising administering to a patient in need of such treatment an effective amount of a compound of formula II: 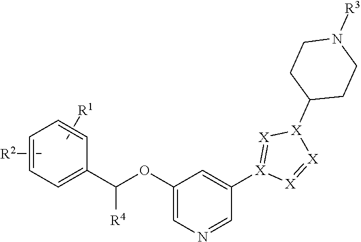
wherein each X is independently C or N;
R1, R2 are independently H, F, Cl, Br or methyl;
R3 is H, amide, carbamate, or alkyl; and
R4 is H or CH₃,
or its pharmaceutically acceptable salt thereof.

15. A method of treatment or prevention of a neurodegenerative disorder or related condition or to inhibit the progression of said neurological disorder or related condition, comprising administering to a patient in need of such treatment an effective amount of a compound of formula III: 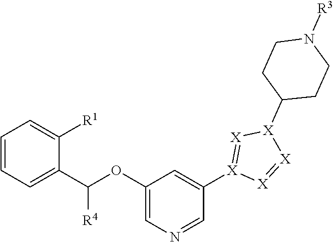
wherein each X is independently C or N;
R1 is H, F, Cl, Br or methyl;
R3 is H, amide, carbamate, or alkyl; and
R4 is H or CH₃,
or its pharmaceutically acceptable salt thereof.

16. A method of treatment or prevention of a neurodegenerative disorder or related condition or to inhibit the progression of said neurological disorder or related condition, comprising administering to a patient in need of such treatment an effective amount of a compound of formula IV: 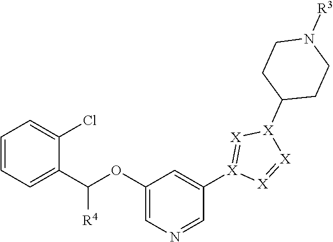
wherein each X is independently C or N;
R3 is H, amide, carbamate, or alkyl; and
R4 is H or CH₃,
or its pharmaceutically acceptable salt thereof.

17. A method of treatment or prevention of a neurodegenerative disorder or related condition or to inhibit the progression of said neurological disorder or related condition, comprising administering to a patient in need of such treatment an effective amount of a compound selected from 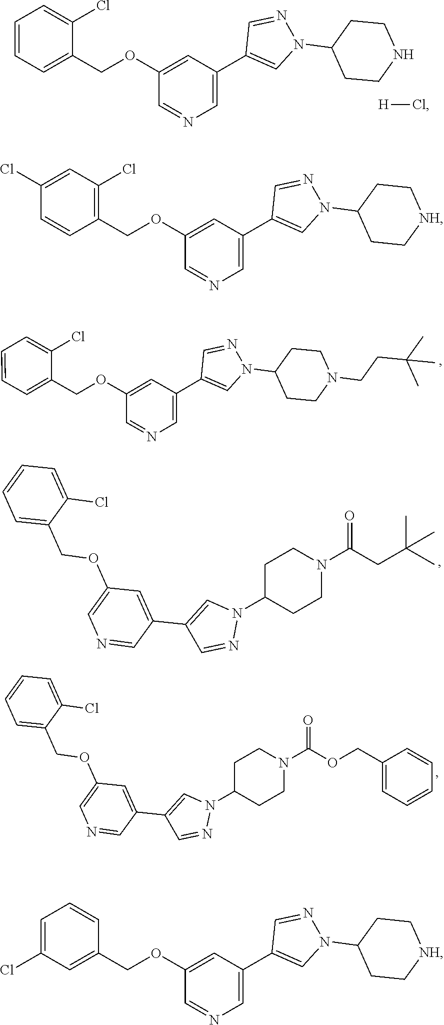 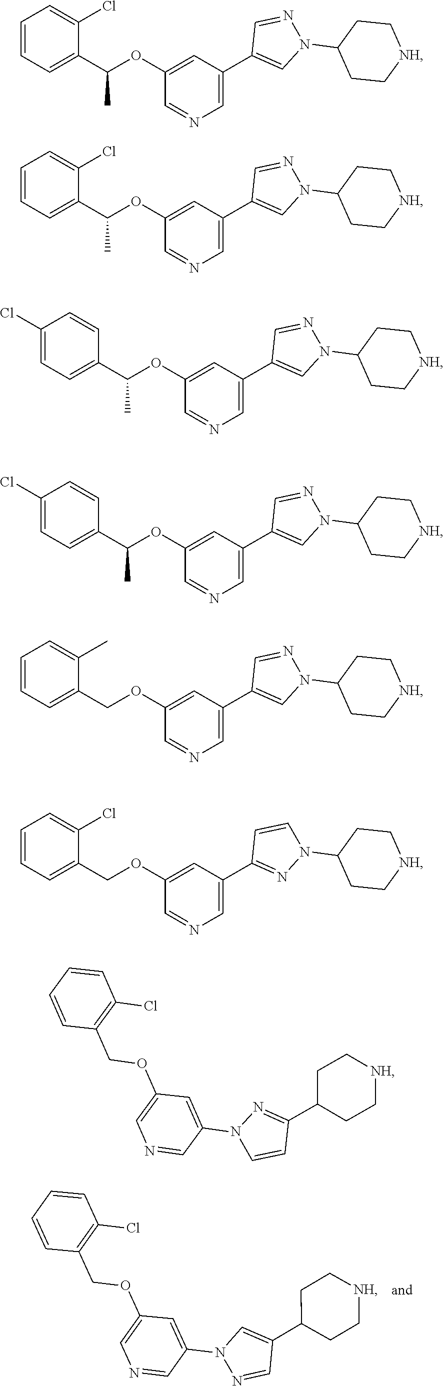 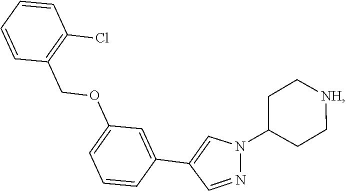 [SMILES: Clc1ccccc1COc1cccc(-c2cnn(C3CCNCC3)c2)c1]
or its pharmaceutically acceptable salt thereof.

18. A method of treatment or prevention of a neurodegenerative disorder or related condition or to inhibit the progression of said neurological disorder or related condition, comprising administering to a patient in need of such treatment an effective amount of compound: 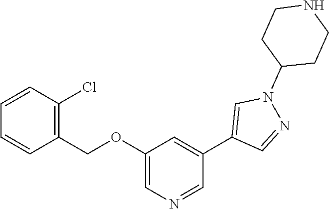 [SMILES: Clc1ccccc1COc1cncc(-c2cnn(C3CCNCC3)c2)c1]
or its pharmaceutically acceptable salt thereof.

19. The method of claim 2, wherein the neurodegenerative disorder or related condition is selected from the group consisting of Alzheimer's disease, Alzheimer's disease-related dementias or mild cognitive impairment.

20. The method of claim 19, wherein the Alzheimer's disease-related dementia is selected from the group consisting of Lewy body dementia (LBD), frontotemporal degeneration (FTD), vascular cognitive impairment and dementia (VCID), and multiple etiology dementias.

21. The pharmaceutical composition of claim 11, wherein the neurodegenerative disorder or related condition is selected from the group consisting of Alzheimer's disease, Alzheimer's disease-related dementias or mild cognitive impairment.

22. The pharmaceutical composition of claim 21, wherein the Alzheimer's disease-related dementia is selected from the group consisting of Lewy body dementia (LBD), frontotemporal degeneration (FTD), vascular cognitive impairment and dementia (VCID), and multiple etiology dementias.

23. The method of claim 14, wherein the neurodegenerative disorder or related condition is selected from the group consisting of Alzheimer's disease, Alzheimer's disease-related dementias or mild cognitive impairment.

24. The method of claim 23, wherein the Alzheimer's disease-related dementia is selected from the group consisting of Lewy body dementia (LBD), frontotemporal degeneration (FTD), vascular cognitive impairment and dementia (VCID), and multiple etiology dementias.

25. A method of synthesizing a compound of formula I: 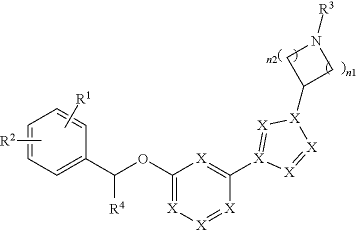
wherein each X is independently C or N;
$$n 1 + n 2 = 4 ;$$
R1, R2 are independently H, F, Cl, Br or methyl;
R3 is H, amide, carbamate, or alkyl; and
R4 is H or CH₃,
or pharmaceutically acceptable salt thereof.

26. The compound of claim 1, wherein the compound of formula I is:

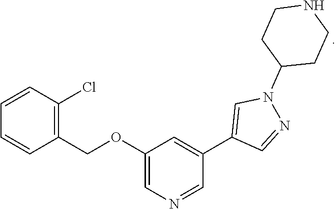 [SMILES: Clc1ccccc1COc1cncc(-c2cnn(C3CCNCC3)c2)c1]

27. The method of claim 15, wherein the neurodegenerative disorder or related condition is selected from the group consisting of Alzheimer's disease, Alzheimer's disease-related dementias or mild cognitive impairment.

28. The method of claim 27, wherein the Alzheimer's disease-related dementia is selected from the group consisting of Lewy body dementia (LBD), frontotemporal degeneration (FTD), vascular cognitive impairment and dementia (VCID), and multiple etiology dementias.

29. The method of claim 16, wherein the neurodegenerative disorder or related condition is selected from the group consisting of Alzheimer's disease, Alzheimer's disease-related dementias or mild cognitive impairment.

30. The method of claim 29, wherein the Alzheimer's disease-related dementia is selected from the group consisting of Lewy body dementia (LBD), frontotemporal degeneration (FTD), vascular cognitive impairment and dementia (VCID), and multiple etiology dementias.

31. The method of claim 17, wherein the neurodegenerative disorder or related condition is selected from the group consisting of Alzheimer's disease, Alzheimer's disease-related dementias or mild cognitive impairment.

32. The method of claim 31, wherein the Alzheimer's disease-related dementia is selected from the group consisting of Lewy body dementia (LBD), frontotemporal degeneration (FTD), vascular cognitive impairment and dementia (VCID), and multiple etiology dementias.

33. The method of claim 18, wherein the neurodegenerative disorder or related condition is selected from the group consisting of Alzheimer's disease, Alzheimer's disease-related dementias or mild cognitive impairment.

34. The method of claim 33, wherein the Alzheimer's disease-related dementia is selected from the group consisting of Lewy body dementia (LBD), frontotemporal degeneration (FTD), vascular cognitive impairment and dementia (VCID), and multiple etiology dementias.


SEQUENCE LISTING (1 sequence)

SEQ ID NO: 1 - protein, 463 aa, unidentified
SEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQG